SEQUENCE LISTING (12 sequences)

SEQ ID NO: 1 - DNA, 83 bp, Artificial Sequence
  Description of Artificial Sequence Synthetic oligonucleotide
gttttagagctagaaatagcaagttaaaataaggctagtccgttatcaacttgaaaaagtggcaccgagtcggtgcttttttt

SEQ ID NO: 2 - protein, 1368 aa, Streptococcus pyogenes [sequence omitted]

SEQ ID NO: 3 - DNA, 23 bp, Artificial Sequence
  Description of Artificial Sequence Synthetic primer
atggactatcatatgcttaccgt

SEQ ID NO: 4 - DNA, 22 bp, Artificial Sequence
  Description of Artificial Sequence Synthetic primer
ttcaagttgataacggactagc

SEQ ID NO: 5 - RNA, 96 nt, Artificial Sequence
  Description of Artificial Sequence Synthetic oligonucleotide
  modified_base (1)..(20): a, c, u, g, unknown or other
nnnnnnnnnnnnnnnnnnnnguuuuagagcuagaaauagcaaguuaaaauaaggcuaguccguuaucaacuugaaaaaguggcaccgagucggugc

SEQ ID NO: 6 - RNA, 96 nt, Artificial Sequence
  Description of Artificial Sequence Synthetic oligonucleotide
  modified_base (1)..(20): a, c, u, g, unknown or other
nnnnnnnnnnnnnnnnnnnnggguuagagcuagaaauagcaaguuaaccuaaggcuaguccguuaucaacuugaaaaaguggcaccgagucggugc

SEQ ID NO: 7 - DNA, 353 bp, Artificial Sequence
  Description of Artificial Sequence Synthetic polynucleotide
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggtcccctccaccccacagtggggttagagctagaaatagcaagttaacctaaggctagtccgttatcaacttgaaaaagtggcaccgagtcggtgcttttttt

SEQ ID NO: 8 - DNA, 106 bp, Artificial Sequence
  Description of Artificial Sequence Synthetic polynucleotide
cttggctttatatatcttgtggaaaggacgaaacaccggtcccctccaccccacagtggggttagagctagaaatagcaagttaacctaaggctagtccgttatca

SEQ ID NO: 9 - DNA, 111 bp, Artificial Sequence
  Description of Artificial Sequence Synthetic polynucleotide
cttggctttatatatcttgtggaaaggacgaaacaccggtagacgcacctccaccccacagtggggttagagctagaaatagcaagttaacctaaggctagtccgttatca

SEQ ID NO: 10 - DNA, 120 bp, Artificial Sequence
  Description of Artificial Sequence Synthetic polynucleotide
ttggctttatatatcttgtggaaaggacgaaacaccggtagacgcggtcacactgatgcagctagtatgcacctccaccccacagtggggttagagctagaaatagcaagttaacctaag

SEQ ID NO: 11 - DNA, 137 bp, Artificial Sequence
  Description of Artificial Sequence Synthetic polynucleotide
ttggctttatatatcttgtggaaaggacgaaacaccggtagacgctgtgacagagccaacacgcagtctcggtcacactgatgcagctagtatgcacctccaccccacagtggggttagagctagaaatagcaagtt

SEQ ID NO: 12 - DNA, 162 bp, Artificial Sequence
  Description of Artificial Sequence Synthetic polynucleotide
ttggctttatatatcttgtggaaaggacgaaacaccggtagacggctagatgaagagcaagcgcatggactgtgacagagccaacacgcagtctcggtcacactgatgcagctagtatgcacctccaccccacagtggggttagagctagaaatagcaagtt

(19) United States
(12) Patent Application Publication
(10) Pub. No.: US 2019/0161743 A1
(43) Pub. Date: May 30, 2019

(54) Self-Targeting Guide RNAs in CRISPR System

(71) Applicant: President and Fellows of Harvard College, Cambridge, MA (US)

(72) Inventors: George M. Church, Brookline, MA (US); Reza Kalhor, East Boston, MA (US); Prashant G. Mali, La Jolla, CA (US)

(21) Appl. No.: 16/099,473

(22) PCT Filed: May 9, 2017
(86) PCT No.: PCT/US17/31640

§ 371 Date: Nov. 7, 2018

Related U.S. Application Data
(60) Provisional application No. 62/333,544, filed on May 9, 2016.

(51) Int. Cl. C12N 9/22; C12N 15/11

(52) U.S. Cl. CPC C12N 9/22; C12N 2310/20; C12N 15/11

(57) ABSTRACT

CRISPR/Cas9 methods are provided where a guide RNA is engineered to self-target and inactivate a nucleic acid encoding the guide RNA itself.

### RELATED APPLICATION DATA

This application claims priority to U.S. Provisional Application No. 62/333,544 filed on May 9, 2017, which is hereby incorporated herein by reference in its entirety for all purposes.

### STATEMENT OF GOVERNMENT INTERESTS

This invention was made with government support under P50 HG005550 awarded by US National Institutes of Health National Human Genome Research Institute. The government has certain rights in the invention.

### BACKGROUND

The CRISPR type II system is a recent development that has been efficiently utilized in a broad spectrum of species. See Friedland, A. E., et al., Heritable genome editing in C. elegans via a CRISPR-Cas9 system. Nat Methods, 2013. 10(8): p. 741-3, Mali, P., et al., RNA-guided human genome engineering via Cas9. Science, 2013. 339(6121): p. 823-6, Hwang, W. Y., et al., Efficient genome editing in zebrafish using a CRISPR-Cas system. Nat Biotechnol, 2013, Jiang, W., et al., RNA-guided editing of bacterial genomes using CRISPR-Cas systems. Nat Biotechnol, 2013, Jinek, M., et al., RNA-programmed genome editing in human cells. eLife, 2013. 2: p. e00471, Cong, L., et al., Multiplex genome engineering using CRISPR/Cas systems. Science, 2013. 339(6121): p. 819-23, Yin, H., et al., Genome editing with Cas9 in adult mice corrects a disease mutation and phenotype. Nat Biotechnol, 2014. 32(6): p. 551-3. CRISPR is particularly customizable because the active form consists of an invariant Cas9 protein and an easily programmable guide RNA (gRNA). See Jinek, M., et al., A programmable dual-RNA-guided DNA endonuclease in adaptive bacterial immunity. Science, 2012. 337(6096): p. 816-21. Of the various CRISPR orthologs, the Streptococcus pyogenes (Sp) CRISPR is the most well-characterized and widely used. The Cas9-gRNA complex first probes DNA for the protospacer-adjacent motif (PAM) sequence (-NGG for Sp Cas9), after which Watson-Crick base-pairing between the gRNA and target DNA proceeds in a ratchet mechanism to form an R-loop. Following formation of a ternary complex of Cas9, gRNA, and target DNA, the Cas9 protein generates two nicks in the target DNA, creating a double-strand break (DSB) that is predominantly repaired by the non-homologous end joining (NHEJ) pathway or, to a lesser extent, template-directed homologous recombination (HR). CRISPR methods are disclosed in U.S. Pat. Nos. 9,023,649 and 8,697,359. See also, Fu et al., Nature Biotechnology, Vol. 32, Number 3, pp. 279-284 (2014). Additional references describing CRISPR-Cas9 systems including nuclease null variants (dCas9) and nuclease null variants functionalized with effector domains such as transcriptional activation domains or repression domains include J. D. Sander and J. K. Joung, Nature biotechnology 32 (4), 347 (2014); P. D. Hsu, E. S. Lander, and F. Zhang, Cell 157 (6), 1262 (2014); L. S. Qi, M. H. Larson, L. A. Gilbert et al., Cell 152 (5), 1173 (2013); P. Mali, J. Aach, P. B. Stranges et al., Nature biotechnology 31 (9), 833 (2013); M. L. Maeder, S. J. Linder, V. M. Cascio t al., Nature methods 10 (10), 977 (2013); P. Perez-Pinera. D. D. Kocak, C. M. Vockley et al., Nature methods 10 (10), 973 (2013); L. A. Gilbert, M. H. Larson, L. Morsut et al., Cell 154 (2), 442 (2013); P. Mali, K. M. Esvelt, and G. M. Church, Nature methods 10 (10), 957 (2013); and K. M. Esvelt, P. Mali, J. L. Braff et al., Nature methods 10 (11), 1116 (2013).

### SUMMARY

Aspects of the present disclosure are directed to modified CRISPR/Cas9 system having Cas9 nuclease and guide RNA (gRNA) scaffolds that enable the Cas9-gRNA complex to target the DNA locus of the gRNA itself. According to one aspect, the modified CRISPR/Cas9 system can act as a “molecular clock” with adjustable speed. According to other aspects, the modified CRISPR/Cas9 system can also be used in general diagnostic or therapeutic CRISPR applications to either eliminate the gRNA carrying loci after the desired task is accomplished or to regulate the amount of gRNA that is produced.

Cas9 is a nuclease that associates with an RNA molecule of a specific sequence and structure, known as the guide RNA (gRNA), to target a specific target DNA locus for digestion. The identity of the target locus is determined by two factors: first, it must contain a protospacer sequence that matches the spacer sequence of the variable region of the gRNA, second it must contain a short protospacer adjacent motif, known as the PAM, adjacent to its protospacer sequence. Unlike the spacer sequence matching part, the PAM sequence does not exist in the gRNA and is exclusive to the target sequence. The nature of the PAM sequence is determined by the Cas9 protein itself.

In standard applications of the CRISPR/Cas9 system, the Cas9 protein and guide RNAs are introduced into the cells by one or multiple DNA vectors. The products of these loci. i.e., the Cas9 protein and the gRNA, combine to form a complex and cut the endogenous target loci that match both the protospacer and the protospacer adjacent motif (PAM) sequences. The gRNA encoding vector itself, however, is not a target of the Cas9/gRNA complex because while the gRNA contains a cognate protospacer sequence it does not contain a PAM sequence adjacent to the protospacer.

According to one aspect, the present disclosure provides modified Cas9 gRNA scaffolds wherein the gRNA encoding locus is targeted by its own gRNA product. In this aspect, the modified guide RNA sequence includes a spacer sequence complementary to a protospacer sequence and a protospacer adjacent motif (PAM) sequence adjacent to the spacer sequence, wherein the spacer sequence is complementary to a target nucleic acid, wherein the modified guide RNA and the Cas9 protein co-localize to the target nucleic acid encoding the gRNA and the Cas9 protein cleaves the target nucleic acid encoding the gRNA to prevent further expression of the guide RNA sequence.

For purposes of the present disclosure, the protospacer sequence may be referred to as the double stranded sequence targeted by the guide RNA spacer sequence. While the guide RNA spacer sequence will bind to one strand of the protospacer sequence, i.e. the complement of the guide RNA spacer, the sequence of the guide RNA spacer may be described with respect to either strand of the protospacer sequence. For example, the guide RNA spacer sequence may be described as being complementary to one strand of the protospacer sequence while the guide RNA spacer sequence may be described as being identical to the other strand of the protospacer sequence. Accordingly, guide RNA spacer sequences may be described as being designed with respect to either strand. Should a guide RNA spacer sequence be described as being identical to a protospacer sequence, it is to be understood that the guide RNA spacer sequence is being designed with respect to the protospacer strand to which it will not bind. In this manner, the resulting guide RNA spacer sequence will bind to the other protospacer strand to which it is complementary.

Target nucleic acid sequences as described herein may be endogenous or exogenous. An endogenous target is one that exists on the genomic (or otherwise endogenous, e.g., mitochondrial) DNA of the host organism in which the system is provided. An exogenous target sequence is one that does not exist on the genomic (or otherwise endogenous, e.g., mitochondrial) DNA of the host organism in which the system is provided. An exogenous target sequence is one that is nonnaturally occurring within the cell and which may be provided as a plasmid introduced to the cell or a transiently transfected DNA element. In an exemplary embodiment, the exogenous target nucleic acid sequence encodes the modified gRNA itself.

A Cas as described herein may be any Cas known to those of skill in the art that may be directed to a target nucleic acid using a guide RNA as known to those of skill in the art. The Cas may be wild type or a homolog or ortholog thereof, such as Cpf1 (See, Zetsche, Bernd et al., Cpf1 Is a Single RNA-Guided Endonuclease of a Class 2 CRISPR-Cas System, Cell. Volume 163, Issue 3, pgs 759-771, hereby incorporated by reference in its entirety). The Cas may be nonnaturally occurring, such as an engineered Cas. The Cas may have one or more nucleolytic domains altered to prevent nucleolytic activity, such as with a Cas nickase or nuclease null or “dead” Cas. Aspects of the present disclosure utilize nicking to effect cutting of one strand of the target nucleic acid. A nuclease null or “dead” Cas may have a nuclease attached thereto to effect cutting, cleaving or nicking of the target nucleic acid. Such nucleases are known to those of skill in the art.

Embodiments of the present disclosure are directed to methods of inactivating a nucleic acid encoding a guide RNA in a cell including introducing into the cell a first foreign nucleic acid encoding a guide RNA sequence including a spacer sequence complementary to a protospacer sequence and a protospacer adjacent motif adjacent to the spacer sequence, wherein the spacer sequence is complementary to a target nucleic acid, introducing into the cell a second foreign nucleic acid encoding a Cas9 protein, wherein the guide RNA sequence and the Cas9 protein are expressed, wherein the guide RNA sequence and the Cas9 protein co-localize to the first foreign nucleic acid and the Cas9 protein cleaves the first foreign nucleic acid sequence to prevent further expression of the guide RNA sequence. In exemplary) embodiments, the guide RNA and the Cas9 protein co-localize to the target nucleic acid and the Cas9 protein cleaves the target nucleic acid. In further exemplary embodiments, the guide RNA and the Cas9 protein co-localize to the guide RNA-encoding DNA and the Cas9 protein cleaves said DNA.

According to certain aspects, the Cas protein may be provided to the cell as a native protein. According to certain aspects, the Cas protein may be provided to the cell as a nucleic acid which is expressed by the cell to provide the Cas protein. According to certain aspects, the expression of the Cas protein in the cell is inducible. According to certain aspects, the guide RNA may be provided to the cell as a native guide RNA. According to certain aspects, the guide RNA may be provided to the cell as a nucleic acid which is expressed by the cell to provide the guide RNA. According to one aspect, a plurality of guide RNAs may be provided to the cell wherein the guide RNAs are directed to a plurality of target nucleic acid sequences.

According to certain aspects, a guide RNA includes a spacer sequence and a tracr mate sequence forming a crRNA, as is known in the art. According to certain aspects, a tracr sequence, as is known in the art, is also used in the practice of methods described herein. According to one aspect, the tracr sequence and the crRNA sequence may be separate or connected by the linker, as is known in the art. According to one aspect, the tracr sequence and the crRNA sequence may be a fusion.

According to one aspect, the guide RNA is provided to the cell by introducing into the cell a first foreign nucleic acid encoding the guide RNA, wherein the guide RNA is expressed. According to one aspect, the Cas protein is expressed by the cell. According to one aspect, the Cas protein is naturally occurring within the cell. According to one aspect, the Cas protein is provided to the cell by introducing into the cell a second foreign nucleic acid encoding the Cas protein, wherein the Cas protein is expressed. The Cas protein and the guide RNA co-localize to the target nucleic acid.

According to one aspect, the Cas protein is an enzymatically active Cas9 protein that is fully enzymatic as is known in the art or a Cas9 protein nickase as is known in the art. According to one aspect, the cell is in vitro, in vivo or ex vivo. According to one aspect, the cell is a eukaryotic cell or prokaryotic cell. According to one aspect, the cell is a bacteria cell, a yeast cell, a fungal cell, a mammalian cell, a human cell, a stem cell, a progenitor cell, a human induced pluripotent stem cell, a plant cell or an animal cell. According to one aspect, the target nucleic acid is genomic DNA, mitochondrial DNA, plasmid DNA, viral DNA, exogenous DNA or cellular RNA.

According to one aspect, the present disclosure is directed to a method of targeting a nucleic acid encoding a guide RNA in a cell including introducing into the cell a first foreign nucleic acid encoding a guide RNA sequence including a spacer sequence and a protospacer adjacent motif (PAM) adjacent to the spacer sequence, wherein the spacer sequence is complementary to a protospacer sequence in the first foreign nucleic acid and to a protospacer sequence in a target nucleic acid sequence of the genomic DNA, introducing into the cell a second foreign nucleic acid encoding a Cas9 protein, wherein the guide RNA sequence and the Cas9 protein are expressed, and wherein the guide RNA sequence and the Cas9 protein co-localize to the first foreign nucleic acid and the Cas9 protein binds or cleaves the first foreign nucleic acid sequence in a site specific manner.

According to another aspect, the present disclosure is directed to a method of targeting a nucleic acid encoding a guide RNA in vitro including providing a first foreign nucleic acid encoding a guide RNA sequence including a spacer sequence and a protospacer adjacent motif (PAM) adjacent to the spacer sequence, wherein the spacer sequence is complementary to a protospacer sequence in the first foreign nucleic acid, providing a second foreign nucleic acid encoding a Cas9 protein, wherein the guide RNA sequence and the Cas9 protein are expressed, and wherein the guide RNA sequence and the Cas9 protein co-localize to the first foreign nucleic acid and the Cas9 protein binds or cleaves the first foreign nucleic acid sequence in a site specific manner.

According to one aspect, the present disclosure is directed to a cell including a first foreign nucleic acid encoding a guide RNA sequence including a spacer sequence and a protospacer adjacent motif (PAM) adjacent to the spacer sequence, wherein the spacer sequence is complementary to a protospacer sequence in the first foreign nucleic acid and a protospacer sequence in a target nucleic acid sequence of the genomic DNA, a second foreign nucleic acid encoding a Cas9 protein, wherein the guide RNA sequence and the Cas9 protein are expressed, and wherein the guide RNA sequence and the Cas9 protein co-localize to the first foreign nucleic acid and the Cas9 protein binds or cleaves the first foreign nucleic acid sequence in a site specific manner.

According to another aspect, the present disclosure is directed to an in vitro CRISPR system including a first foreign nucleic acid encoding a guide RNA sequence including a spacer sequence and a protospacer adjacent motif (PAM) adjacent to the spacer sequence, wherein the spacer sequence is complementary to a protospacer sequence in the first foreign nucleic acid, a second foreign nucleic acid encoding a Cas9 protein, wherein the guide RNA sequence and the Cas9 protein are expressed, and wherein the guide RNA sequence and the Cas9 protein co-localize to the first foreign nucleic acid and the Cas9 protein binds or cleaves the first foreign nucleic acid sequence in a site specific manner.

According to still another aspect, the present disclosure is directed to a method of targeting a nucleic acid sequence using a CRISPR system including providing a first foreign nucleic acid encoding a guide RNA sequence including a spacer sequence complementary to a protospacer sequence in the nucleic acid sequence, providing a second foreign nucleic acid encoding a Cas9 protein, wherein the guide RNA sequence and the Cas9 protein are expressed, wherein the guide RNA sequence and the Cas9 protein co-localize to the nucleic acid sequence and the Cas9 protein binds or cleaves the nucleic acid sequence in a site specific manner, and wherein the rate at which the guide RNA regulates the binding or cleavage of the nucleic acid sequence can be controlled.

Further features and advantages of certain embodiments of the present invention will become more fully apparent in the following description of embodiments and drawings thereof, and from the claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The foregoing and other features and advantages of the present embodiments will be more fully understood from the following detailed description of illustrative embodiments taken in conjunction with the accompanying drawings in which:

FIG. 1 is a schematic showing a standard application of the CRISPR/Cas9 system. The Cas9 protein and guide RNAs are introduced by DNA vectors into a cell where the Cas9 protein and the gRNA form a complex and cut endogenous target loci that match both the protospacer and the PAM. The vector encoding the guide RNA, however, is not a target of the Cas9/gRNA complex because while it contains a cognate protospacer sequence it does not contain a PAM sequence adjacent to the protospacer.

FIG. 2 is a schematic showing an exemplary application of the self-targeting CRISPR/Cas9 system. The Cas9 protein and modified guide RNAs containing both a cognate protospacer sequence and a PAM sequence adjacent to the protospacer are introduced by DNA vectors into a cell where the Cas9 protein and the modified gRNA form a complex and cut the vector encoding the guide RNA.

FIG. 3 shows sequence comparison between a standard guide RNA and a modified self-targeting guide RNA.

FIG. 4 is a schematic showing that standard gRNAs can only create one alteration in their target sequence in cells because they do not match their target after the target sequence is altered, likely by NHEJ repair.

FIG. 5 is a schematic showing that a self-targeting guide RNA can attack its own encoding locus over and over because after each alteration of the encoding locus the guide RNA that is expressed carries the new protospacer sequence which will match the new sequence of the guide RNA encoding locus.

FIG. 6 shows an exemplary sequence of a self-targeting guide RNA under U6 promoter. The self-targeting guide RNA has an AAVSI-TI protospacer and SpCas9 gRNA scaffold followed by U6 terminator.

FIG. 7 shows the non-reference sequence abundance of the self-targeting guide RNA locus in cells upon induction of Cas9 protein over time.

FIG. 8 shows the accumulation of inactive guide RNA locus in cells upon induction of Cas9 protein over time.

FIG. 9 shows exemplary sequences of five self-targeting guide RNAs under U6 promoter where each guide RNA has a difference distance between its transcription start site and guide RNA scaffold, with Ins0 representing the shortest distance and Ins100 representing the longest distance.

FIG. 10 shows the non-reference sequence abundance of guide RNA locus in cells upon induction of Cas9 protein over time for the five exemplary self-targeting guide RNAs.

FIG. 11 shows the accumulation of inactive guide RNA locus in cells upon induction of Cas9 protein over time for the five exemplary self-targeting guide RNAs.

### DETAILED DESCRIPTION

Embodiments of the present disclosure are directed to modified CRISPR/Cas9 system having Cas9 nuclease and guide RNA (gRNA) scaffolds that enable the Cas9-gRNA complex to target the DNA locus of the gRNA itself. The modified guide RNA sequence includes a spacer sequence complementary to a protospacer sequence and a protospacer adjacent motif (PAM) sequence adjacent to the spacer sequence, wherein the spacer sequence is complementary to a target nucleic acid, wherein the modified guide RNA and the Cas9 protein co-localize to the target nucleic acid encoding the gRNA and the Cas9 protein cleaves the target nucleic acid encoding the gRNA to prevent further expression of the guide RNA sequence.

Methods described herein can be used to cleave exogenous nucleic acids. Methods described herein can be used to cleave endogenous nucleic acids. Methods described herein can be used with known Cas proteins or orthologs or engineered versions thereof. Methods described herein can be practiced in vivo, ex vivo or in vitro. Methods described herein can be multiplexed within a single target nucleic acid region or across multiple regions.

According to one aspect, the present disclosure provides a method of targeting a nucleic acid encoding a guide RNA in a cell. The method includes introducing into the cell a first foreign nucleic acid encoding a guide RNA sequence including a spacer sequence and a protospacer adjacent motif (PAM) adjacent to the spacer sequence, wherein the spacer sequence is complementary to a protospacer sequence in the first foreign nucleic acid and to a protospacer sequence in a target nucleic acid sequence of the genomic DNA, introducing into the cell a second foreign nucleic acid encoding a Cas9 protein, wherein the guide RNA sequence and the Cas9 protein are expressed, and wherein the guide RNA sequence and the Cas9 protein co-localize to the first foreign nucleic acid and the Cas9 protein binds or cleaves the first foreign nucleic acid sequence in a site specific manner. In one embodiment, the guide RNA and the Cas9 protein form a co-localization complex at the first foreign nucleic acid sequence, wherein the binding or cleaving of the first foreign nucleic acid sequence alters the expression of the guide RNA or inactivates the first foreign nucleic acid sequence encoding the guide RNA.

In another embodiment, the guide RNA and the Cas9 protein co-localize to the target nucleic acid sequence and the Cas9 protein binds or cleaves the target nucleic acid sequence in a site specific manner. In one embodiment, the binding or cleaving of the target nucleic acid sequence alters the expression of the target nucleic acid sequence.

In certain embodiments, the first foreign nucleic acid sequence that is cleaved in a site specific manner is repaired by non-homologous end joining repair mechanism to form a repaired subsequent foreign nucleic acid sequence encoding a subsequent guide RNA having a subsequent spacer sequence complementary to a subsequent target nucleic acid sequence of the genomic DNA. In some embodiments, the repaired subsequent foreign nucleic acid sequence is expressed to form the subsequent guide RNA which forms a colocalization complex with the Cas9 protein and the repaired subsequent foreign nucleic acid sequence, wherein the Cas9 protein cleaves the repaired subsequent foreign nucleic acid sequence in a site specific manner to prevent further expression of the subsequent guide RNA sequence. In certain other embodiments, the subsequent guide RNA and the Cas9 protein co-localize to the subsequent target nucleic acid sequence and the Cas9 protein cleaves the subsequent target nucleic acid sequence in a site specific manner. In some embodiments, the process of cleaving the first foreign nucleic acid sequence, repairing the first foreign nucleic acid sequence, expressing the repaired subsequent foreign nucleic acid sequence, cleaving the repaired subsequent foreign nucleic acid sequence in a site specific manner, and cleaving the subsequent target nucleic acid sequence in a site specific manner is cycled in the cell to result in (1) eliminating or inactivating the foreign nucleic acid sequence and (2) a plurality of target nucleic acid sequences being cleaved.

The Cas protein according to certain embodiments of the present disclosure includes a Type II CRISPR system Cas9 protein or its ortholog such as Cpf1. The Cas9 protein according to certain embodiments of the present disclosure includes an enzymatically active Cas9 protein having nuclease activity that can cut both strands of the target nucleic acid, a Cas9 protein nickase that cuts one strand of the target nucleic acid, or a nuclease null Cas9 protein or “dead” Cas9 protein. The nuclease null Cas9 protein and the guide RNA colocalize to the target nucleic acid or the nucleic acid encoding the guide RNA resulting in binding but not cleaving of the target nucleic acid or the nucleic acid encoding the guide RNA. The activity or transcription of the target nucleic acid or the nucleic acid encoding the guide RNA is regulated by such binding. The Cas9 protein can further comprise a transcriptional regulator or DNA modifying protein attached thereto. Exemplary transcriptional regulators are known to a skilled in the art and include VPR, VP64, P65 and RTA. Exemplary DNA-modifying enzymes are known to a skilled in the art and include Cytidine deaminases, APOBECs, Fok1, endonucleases and DNases. Binding but not cleaving can occur in circumstances where a guide RNA having a shortened spacer sequence is used with an enzymatically active Cas9 protein, which is known the art and has been described in Kiani S, Chavez A, Tuttle M. Hall R N, Chari R, Ter-Ovanesyan D, Qian J. Pruitt B W, Beal J, Vora S, Buchthal J, Kowal E J, Ebrahimkhani M R, Collins J J, Weiss R. Church G, Cas9 gRNA engineering for genome editing, activation and repression, Nat Methods., 2015 November; 12(11):1051-4, Epub 2015 Sep. 7; and Chavez A, Scheiman J, Vora S, Pruitt B W, Tuttle M, P R Iyer E. Lin S, Kiani S, Guzman C D, Wiegand D J, Ter-Ovanesyan D. Braff J L, Davidsohn N, Housden B E, Perrimon N, Weiss R, Aach J, Collins J J, Church G M., Highly efficient Cas9-mediated transcriptional programming, Nat Methods., 2015 April; 12(4):326-8, Epub 2015 Mar. 2, each of which are hereby incorporated by reference in its entirety.

The cell according to certain embodiments of the present disclosure includes a eukaryotic cell or prokaryotic cell. In some embodiments, the cell is a bacteria cell, yeast cell, a mammalian cell, a human cell, a plant cell or an animal cell.

In one embodiment, the rate at which the guide RNA regulates the binding or cleavage of the first foreign nucleic acid sequence and/or the target nucleic acid sequence can be controlled by adding additional nucleotide sequence between the transcription start site and the scaffold of the guide RNA. In another embodiment, increasing the length of the additional nucleotide sequence between the transcription start site and the scaffold of the guide RNA reduces the rate at which the guide RNA regulates the binding or cleavage of the first foreign nucleic acid sequence and/or the target nucleic acid sequence.

Methods described herein can be used for cellular and molecular barcoding. Methods described herein can be used to measure and record various cellular events that are coupled to production of the Cas9 protein or the guide RNA. The cellular events include cell divisions, lineage tracing and cellular signaling.

In some embodiments, the first and/or the second foreign nucleic acid sequence are exogenous to the cell. In other embodiments, the first and/or the second foreign nucleic acid sequence are integrated into the cell's genomic DNA.

In certain exemplary embodiments, the activity or expression of the Cas9 protein is inducible. In some embodiments, the native Cas9 protein instead of the nucleic acid encoding the Cas9 protein is introduced to the cell.

According to another aspect, the present disclosure provides a method of targeting a nucleic acid encoding a guide RNA in vitro. The method includes providing a first foreign nucleic acid encoding a guide RNA sequence including a spacer sequence and a protospacer adjacent motif (PAM) adjacent to the spacer sequence, wherein the spacer sequence is complementary to a protospacer sequence in the first foreign nucleic acid, providing a second foreign nucleic acid encoding a Cas9 protein, wherein the guide RNA sequence and the Cas9 protein are expressed, and wherein the guide RNA sequence and the Cas9 protein co-localize to the first foreign nucleic acid and the Cas9 protein binds or cleaves the first foreign nucleic acid sequence in a site specific manner.

In one embodiment, the binding or cleaving of the first foreign nucleic acid sequence alters the expression of the guide RNA or inactivates the first foreign nucleic acid sequence encoding the guide RNA. In another embodiment, other DNA having a target nucleic acid sequence is further provided, wherein the spacer sequence of the guide RNA is complementary to a protospacer sequence in the target nucleic acid sequence, and wherein the guide RNA and the Cas9 protein co-localize to the target nucleic acid sequence and the Cas9 protein binds or cleaves the target nucleic acid sequence in a site specific manner. In one embodiment, the binding or cleaving of the target nucleic acid sequence alters the expression of the target nucleic acid sequence.

In some embodiments, the Cas9 is a Type II CRISPR system Cas9 or Cpf1. In other embodiments, the Cas9 protein is an enzymatically active Cas9 protein, a Cas9 protein nickase, or a nuclease null Cas9 protein. In still other embodiments, the Cas9 protein further comprises a transcriptional regulator or a DNA modifying protein attached thereto.

In some embodiments, the guide RNA instead of the nucleic acid encoding the guide RNA is provided. In some embodiments, the native Cas9 protein instead of the nucleic acid encoding the Cas9 protein is provided.

In certain exemplary embodiments, the rate at which the guide RNA regulates the binding or cleavage of the first foreign nucleic acid sequence and/or the target nucleic acid sequence can be controlled by adding additional nucleotide sequence between the transcription start site and the scaffold of the guide RNA. In certain embodiments, increasing the length of the additional nucleotide sequence between the transcription start site and the scaffold of the guide RNA reduces the rate at which the guide RNA regulates the binding or cleavage of the first foreign nucleic acid sequence and/or the target nucleic acid sequence. In some embodiments, the length of the additional nucleotide sequence is between about 5 and about 500 nucleotides, between about 10 and about 200 nucleotides, between about 20 and about 100 nucleotides, between about 30 and about 90 nucleotides, between about 40 and about 80 nucleotides, between about 50 and about 70 nucleotides and between about 55 and about 65 nucleotides long.

Methods described herein can be used for molecular cloning and genetic engineering applications. For instance, methods described herein can be used to remove exogenous sequences of DNA that are inserted into cells and to target genes for therapeutic purposes. Methods described herein can be used to deplete or enrich specific targets in a library of DNA molecules. For instance, methods described herein can be used to cut a specific set of target molecules in a library of DNA molecules.

In some embodiments, the first and/or the second foreign nucleic acid sequence are genomic DNA or exogenous to the genomic DNA. In some other embodiments, the first and/or the second foreign nucleic acid sequence are integrated into the genomic DNA.

In certain embodiments, the activity or expression of the Cas9 protein is inducible.

According to one aspect, the present disclosure provides a cell including a first foreign nucleic acid encoding a guide RNA sequence including a spacer sequence and a protospacer adjacent motif (PAM) adjacent to the spacer sequence, wherein the spacer sequence is complementary to a protospacer sequence in the first foreign nucleic acid and a protospacer sequence in a target nucleic acid sequence of the genomic DNA, a second foreign nucleic acid encoding a Cas9 protein, wherein the guide RNA sequence and the Cas9 protein are expressed, and wherein the guide RNA sequence and the Cas9 protein co-localize to the first foreign nucleic acid and the Cas9 protein binds or cleaves the first foreign nucleic acid sequence in a site specific manner. In some embodiments, the binding or cleaving of the first foreign nucleic acid sequence alters the expression of the guide RNA or inactivates the first foreign nucleic acid sequence encoding the guide RNA. In some other embodiments, the guide RNA and the Cas9 protein co-localize to the target nucleic acid sequence and the Cas9 protein binds or cleaves the target nucleic acid sequence in a site specific manner.

The cell according to certain embodiments of the present disclosure includes a eukaryotic cell or prokaryotic cell. In some embodiments, the cell is a bacteria cell, yeast cell, a mammalian cell, a human cell, a plant cell or an animal cell.

In some embodiments, the first and/or the second foreign nucleic acid sequence are exogenous to the cell. In other embodiments, the first and/or the second foreign nucleic acid sequence are integrated into the cell's genomic DNA. In certain embodiments, the activity or expression of the Cas9 protein is inducible.

According to one aspect, the present disclosure provides an in vitro CRISPR system including a first foreign nucleic acid encoding a guide RNA sequence including a spacer sequence and a protospacer adjacent motif (PAM) adjacent to the spacer sequence, wherein the spacer sequence is complementary to a protospacer sequence in the first foreign nucleic acid, a second foreign nucleic acid encoding a Cas9 protein, wherein the guide RNA sequence and the Cas9 protein are expressed, and wherein the guide RNA sequence and the Cas9 protein co-localize to the first foreign nucleic acid and the Cas9 protein binds or cleaves the first foreign nucleic acid sequence in a site specific manner.

In some embodiments, the binding or cleaving of the first foreign nucleic acid sequence alters the transcription of the guide RNA or inactivates the first foreign nucleic acid sequence encoding the guide RNA. In other embodiments, the in vitro CRISPR system further includes a DNA library having a target nucleic acid sequence, wherein the spacer sequence of the guide RNA is complementary to a protospacer sequence in the target nucleic acid sequence, and wherein the guide RNA and the Cas9 protein co-localize to the target nucleic acid sequence and the Cas9 protein binds or cleaves the target nucleic acid sequence in a site specific manner. In some embodiments, the binding or cleaving of the target nucleic acid sequence alters the activity of the target nucleic acid sequence.

In some embodiments, the Cas9 is a Type II CRISPR system Cas9 or Cpf1. In other embodiments, the Cas9 protein is an enzymatically active Cas9 protein, a Cas9 protein nickase, or a nuclease null Cas9 protein. In certain embodiments, the Cas9 protein further includes a transcriptional regulator or a DNA-modifying protein attached thereto.

In some embodiments, the guide RNA instead of the nucleic acid encoding the guide RNA is provided. In other embodiments, the Cas9 protein instead of the nucleic acid encoding the Cas9 protein is provided.

The in vitro CRISPR system as described herein, wherein the rate at which the guide RNA regulates the binding or cleavage of the first foreign nucleic acid sequence and/or the target nucleic acid sequence can be controlled by adding additional nucleotide sequence between the transcription start site and the scaffold of the guide RNA. The in vitro CRISPR system as described herein, wherein increasing the length of the additional nucleotide sequence between the transcription start site and the scaffold of the guide RNA reduces the rate at which the guide RNA regulates the binding or cleavage of the first foreign nucleic acid sequence and/or the target nucleic acid sequence.

In some embodiments, the first and/or the second foreign nucleic acid sequence are a library of DNA molecules. In other embodiments, the first and/or the second foreign nucleic acid sequence are integrated into the library of DNA molecules. In certain embodiments, the activity or expression of the Cas9 protein is inducible.

According to another aspect, the present disclosure provides a method of targeting a nucleic acid sequence using a CRISPR system including providing a first foreign nucleic acid encoding a guide RNA sequence including a spacer sequence complementary to a protospacer sequence in the nucleic acid sequence, providing a second foreign nucleic acid encoding a Cas9 protein, wherein the guide RNA sequence and the Cas9 protein are expressed, wherein the guide RNA sequence and the Cas9 protein co-localize to the nucleic acid sequence and the Cas9 protein binds or cleaves the nucleic acid sequence in a site specific manner, and wherein the rate at which the guide RNA regulates the binding or cleavage of the nucleic acid sequence can be controlled. In some embodiments, the rate at which the guide RNA regulates the binding or cleavage of the nucleic acid sequence can be controlled by adding additional nucleotide sequence between the transcription start site and the scaffold of the guide RNA.

Methods described herein can target the nucleic acid sequence in a cell or in vitro.

In certain exemplary embodiments, the nucleic acid sequence encodes a self-targeting guide RNA including a spacer sequence and a protospacer adjacent motif (PAM) adjacent to the spacer sequence, wherein the spacer sequence is complementary to a protospacer sequence in the nucleic acid. In some embodiments, the rate at which the self-targeting guide RNA regulates the binding or cleavage of the nucleic acid sequence can be controlled by adding additional nucleotide sequence between the transcription start site and the scaffold of the guide RNA. In other embodiments, increasing the length of the additional nucleotide sequence between the transcription start site and the scaffold of the guide RNA reduces the rate at which the guide RNA regulates the binding or cleavage of the first foreign nucleic acid sequence and/or the target nucleic acid sequence.

According to certain aspects, an exemplary spacer sequence is between 10 and 30 nucleotides in length. According to certain aspects, an exemplary spacer sequence is between 15 and 25 nucleotides in length. An exemplary spacer sequence is between 18 and 22 nucleotides in length. An exemplary spacer sequence is 20 nucleotides in length. According to certain methods, two or more or a plurality of guide RNAs may be used in the practice of certain embodiments.

The term spacer sequence is understood by those of skill in the art and may include any polynucleotide having sufficient complementarity with a target nucleic acid sequence to hybridize with the target nucleic acid sequence and direct sequence-specific binding of a CRISPR complex to the target sequence. A CRISPR complex may include the guide RNA and the Cas9 protein. The guide RNA may be formed from a spacer sequence covalently connected to a tracr mate sequence (which may be referred to as a crRNA) and a separate tracr sequence, wherein the tracr mate sequence is hybridized to a portion of the tracr sequence. According to certain aspects, the tracr mate sequence and the tracr sequence are connected or linked such as by covalent bonds by a linker sequence, which construct may be referred to as a fusion of the tracr mate sequence and the tracr sequence. The linker sequence referred to herein is a sequence of nucleotides, referred to herein as a nucleic acid sequence, which connect the tracr mate sequence and the tracr sequence. Accordingly, a guide RNA may be a two component species (i.e., separate crRNA and tracr RNA which hybridize together) or a unimolecular species (i.e., a crRNA-tracr RNA fusion, often termed an sgRNA).

Tracr mate sequences and tracr sequences are known to those of skill in the art, such as those described in US 2014/0356958. The tracr mate sequence and tracr sequence used in the present disclosure is N20 to N8-gttttagagctagaaatagcaagttaaaataaggctagtccgttatcaacttgaaagtggcaccgagtcggtgcttttttt with N20-8 being the number of nucleotides complementary to a target locus of interest.

According to certain aspects, the tracr mate sequence is between about 17 and about 27 nucleotides in length. According to certain aspects, the tracr sequence is between about 65 and about 75 nucleotides in length. According to certain aspects, the linker nucleic acid sequence is between about 4 and about 6.

According to one aspect, embodiments described herein include guide RNA having a length including the sum of the lengths of a spacer sequence, tracr mate sequence, tracr sequence, and linker sequence (if present). Accordingly, such a guide RNA may be described by its total length which is a sum of its spacer sequence, tracr mate sequence, tracr sequence, and linker sequence. According to this aspect, all of the ranges for the spacer sequence, tracr mate sequence, tracr sequence, and linker sequence (if present) are incorporated herein by reference and need not be repeated. One of skill will readily be able to sum each of the portions of a guide RNA to obtain the total length of the guide RNA sequence. Aspects of the present disclosure are directed to methods of making such guide RNAs as described herein by expressing constructs encoding such guide RNA using promoters and terminators and optionally other genetic elements as described herein.

According to certain aspects, the cell includes a naturally occurring Cas protein. According to certain aspects, the guide RNA and the Cas protein which interacts with the guide RNA are foreign to the cell into which they are introduced or otherwise provided. According to this aspect, the guide RNA and the Cas protein are nonnaturally occurring in the cell in which they are introduced, or otherwise provided. To this extent, cells may be genetically engineered or genetically modified to include the CRISPR/Cas systems described herein.

Exemplary Cas protein include S. pyogenes Cas9, S. thermophilus Cas9 and S. aureus Cas9. One exemplary CRISPR/Cas system uses the S. pyogenes Cas9 nuclease (Sp. Cas9), an extremely high-affinity (see Stemberg, S. H., Redding, S., Jinek, M., Greene, E. C. & Doudna. J. A. DNA interrogation by the CRISPR RNA-guided endonuclease Cas9. Nature 507, 62-67 (2014) hereby incorporated by reference in its entirety), programmable DNA-binding protein isolated from a type II CRISPR-associated system (see Gameau. J. E. et al. The CRISPR/Cas bacterial immune system cleaves bacteriophage and plasmid DNA. Nature 468, 67-71 (2010) and Jinek, M. et al. A programmable dual-RNA-guided DNA endonuclease in adaptive bacterial immunity. Science 337, 816-821 (2012) each of which are hereby incorporated by reference in its entirety). The DNA locus targeted by Cas9 precedes a three nucleotide (nt) 5′-NGG-3′ “PAM” sequence, and matches a 15-22-nt guide or spacer sequence within a Cas9-bound RNA cofactor, referred to herein and in the art as a guide RNA. Altering this guide RNA is sufficient to target Cas9 to a target nucleic acid. In a multitude of CRISPR-based biotechnology applications, the guide is often presented in a so-called sgRNA (single guide RNA), wherein the two natural Cas9 RNA cofactors (gRNA and tracrRNA) are fused via an engineered loop.

Embodiments of the present disclosure are directed to methods of inactivating a nucleic acid encoding a guide RNA in a cell including introducing into the cell a first foreign nucleic acid encoding a guide RNA sequence including a spacer sequence complementary to a protospacer sequence and a protospacer adjacent motif adjacent to the spacer sequence, wherein the spacer sequence is complementary to a target nucleic acid, introducing into the cell a second foreign nucleic acid encoding a Cas9 protein, wherein the guide RNA sequence and the Cas9 protein are expressed, wherein the guide RNA sequence and the Cas9 protein co-localize to the first foreign nucleic acid and the Cas9 protein cleaves the first foreign nucleic acid sequence to prevent further expression of the guide RNA sequence. In exemplary embodiments, the guide RNA and the Cas9 protein co-localize to the target nucleic acid and the Cas9 protein cleaves the target nucleic acid. Methods described herein can be performed in vitro, in vivo or ex vivo. According to one aspect, the cell is a eukaryotic cell or a prokaryotic cell. According to one aspect, the cell is a bacteria cell, a yeast cell, a mammalian cell, a human cell, a stem cell, a progenitor cell, an induced pluripotent stem cell, a human induced pluripotent stem cell, a plant cell or an animal cell. According to one aspect, the Cas9 protein is an enzymatically active Cas9 protein, a Cas9 protein wild-type protein, or an enzymatically active Cas9 nickase. Additional exemplary Cas9 proteins include Cas9 proteins attached to, bound to or fused with a nuclease such as a Fok-domain, such as Fok 1 and the like. Exemplary nucleases are known to those of skill in the art.

According to certain aspects, the Cas protein may be delivered directly to a cell as a native species by methods known to those of skill in the art, including injection or lipofection, or as translated from its cognate mRNA, or transcribed from its cognate DNA into mRNA (and thereafter translated into protein). Cas DNA and mRNA may be themselves introduced into cells through electroporation, transient and stable transfection (including lipofection) and viral transduction or other methods known to those of skill in the art. According to certain aspects, the guide RNA may be delivered directly to a cell as a native species by methods known to those of skill in the art, including injection or lipofection, or as transcribed from its cognate DNA, with the cognate DNA introduced into cells through electroporation, transient and stable transfection (including lipofection) and viral transduction.

According to certain aspects, a first foreign nucleic acid encoding a guide RNA sequence including a spacer sequence complementary to a protospacer sequence and a protospacer adjacent motif adjacent to the spacer sequence is provided to a cell. The spacer sequence is complementary to a target nucleic acid. A second foreign nucleic acid encoding a Cas9 protein is provided to the cell. The cell expresses the guide RNA sequence and the Cas9 protein, wherein the guide RNA sequence and the Cas9 protein co-localize to the first foreign nucleic acid and the Cas9 protein cleaves the first foreign nucleic acid sequence to prevent further expression of the guide RNA sequence. In exemplary embodiments, the guide RNA and the Cas9 protein co-localize to the target nucleic acid and the Cas9 protein cleaves the target nucleic acid. The cell may be any desired cell including a eukaryotic cell. An exemplary cell is a human cell.

Cas9 proteins and Type II CRISPR systems are well documented in the art. See Makarova et al., Nature Reviews, Microbiology, Vol. 9, June 2011, pp. 467-477 including all supplementary information hereby incorporated by reference in its entirety. In general, bacterial and archaeal CRISPR-Cas systems rely on short guide RNAs in complex with Cas proteins to direct degradation of complementary sequences present within invading foreign nucleic acid. See Deltcheva, E. et al. CRISPR RNA maturation by trans-encoded small RNA and host factor RNase III. Nature 471, 602-607 (2011); Gasiunas, G., Barrangou, R., Horvath. P. & Siksnys, V. Cas9-crRNA ribonucleoprotein complex mediates specific DNA cleavage for adaptive immunity in bacteria. Proceedings of the National Academy of Sciences of the United States of America 109, E2579-2586 (2012); Jinek, M. et al. A programmable dual-RNA-guided DNA endonuclease in adaptive bacterial immunity. Science 337, 816-821 (2012); Sapranauskas, R. et al. The Streptococcus thermophilus CRISPR/Cas system provides immunity in Escherichia coli. Nucleic acids research 39, 9275-9282 (2011); and Bhaya. D., Davison, M. & Barrangou, R. CRISPR-Cas systems in bacteria and archaea: versatile small RNAs for adaptive defense and regulation. Annual review of genetics 45, 273-297 (2011). A recent in vitro reconstitution of the S. pyogenes type II CRISPR system demonstrated that crRNA (“CRISPR RNA”) fused to a normally trans-encoded tracrRNA (“trans-activating CRISPR RNA”) is sufficient to direct Cas9 protein to sequence-specifically cleave target DNA sequences matching the crRNA. Expressing a gRNA homologous to a target site results in Cas9 recruitment and degradation of the target DNA. See H. Deveau et al., Phage response to CRISPR-encoded resistance in Streptococcus thermophilus. Journal of Bacteriology 190, 1390 (February 2008).

Three classes of CRISPR systems are generally known and are referred to as Type I, Type II or Type III). According to one aspect, a particular useful enzyme according to the present disclosure to cleave dsDNA is the single effector enzyme, Cas9, common to Type II. See K. S. Makarova et al., Evolution and classification of the CRISPR-Cas systems. Nature reviews. Microbiology 9, 467 (June, 2011) hereby incorporated by reference in its entirety. Within bacteria, the Type 11 effector system consists of a long pre-crRNA transcribed from the spacer-containing CRISPR locus, the multifunctional Cas9 protein, and a tracrRNA important for gRNA processing. The tracrRNAs hybridize to the repeat regions separating the spacers of the pre-crRNA, initiating dsRNA cleavage by endogenous RNase III, which is followed by a second cleavage event within each spacer by Cas9, producing mature crRNAs that remain associated with the tracrRNA and Cas9. TracrRNA-crRNA fusions are contemplated for use in the present methods.

According to one aspect, the enzyme of the present disclosure, such as Cas9 unwinds the DNA duplex and searches for sequences matching the crRNA to cleave. Target recognition occurs upon detection of complementarity between a “protospacer” sequence in the target DNA and the remaining spacer sequence in the crRNA. Importantly, Cas9 cuts the DNA only if a correct protospacer-adjacent motif (PAM) is also present at the 3′ end.

According to certain aspects, different protospacer-adjacent motif can be utilized. For example, the S. pyogenes system requires an NGG sequence, where N can be any nucleotide. S. thermophilus Type II systems require NGGNG (see P. Horvath. R. Barrangou. CRISPR/Cas, the immune system of bacteria and archaea. Science 327, 167 (Jan. 8, 2010) hereby incorporated by reference in its entirety and NNAGAAW (see H. Deveau et al., Phage response to CRISPR-encoded resistance in Streptococcus thermophilus. Journal of bacteriology 190, 1390 (February, 2008) hereby incorporated by reference in its entirety), respectively, while different S. mutans systems tolerate NGG or NAAR (see J. R. van der Ploeg, Analysis of CRISPR in Streptococcus mutans suggests frequent occurrence of acquired immunity against infection by M102-like bacteriophages. Microbiology 155, 1966 (June, 2009) hereby incorporated by reference in its entirety. Bioinformatic analyses have generated extensive databases of CRISPR loci in a variety of bacteria that may serve to identify additional useful PAMs and expand the set of CRISPR-targetable sequences (see M. Rho, Y. W. Wu, H. Tang, T. G. Doak, Y. Ye, Diverse CRISPRs evolving in human microbiomes. PLoS genetics 8, e1002441 (2012) and D. T. Pride et al., Analysis of streptococcal CRISPRs from human saliva reveals substantial sequence diversity within and between subjects over time. Genome research 21, 126 (January, 2011) each of which are hereby incorporated by reference in their entireties.

In S. pyogenes, Cas9 generates a blunt-ended double-stranded break 3 bp upstream of the protospacer-adjacent motif (PAM) via a process mediated by two catalytic domains in the protein: an HNH domain that cleaves the complementary strand of the DNA and a RuvC-like domain that cleaves the non-complementary strand. See Jinek et al., Science 337, 816-821 (2012) hereby incorporated by reference in its entirety. Cas9 proteins are known to exist in many Type II CRISPR systems including the following as identified in the supplementary information to Makarova et al., Nature Reviews, Microbiology, Vol. 9, June 2011, pp. 467-477: Methanococcus maripaludis C7; Corynebacterium diphtheriae; Corynebacterium efficiens YS-314; Corynebacterium glutamicum ATCC 13032 Kitasato; Corynebacterium glutamicum ATCC 13032 Bielefeld; Corynebacterium glutamicum R; Corynebacterium kroppenstedtii DSM 44385; Mycobacterium abscessus ATCC 19977; Nocardia farcinica IFM10152; Rhodococcus erythropolis PR4; Rhodococcus jostii RHA 1; Rhodococcus opacus B4 uid36573; Acidothermus cellulolyticus 11B; Arthrobacter chlorophenolicus A6; Kribbella flavida DSM 17836 uid43465; Thermomonospora curvata DSM 43183; Bifidobacterium dentium Bd1; Bifidobacterium longum DJO10A; Slackia heliotrinireducens DSM 20476; Persephonella marina EX H1; Bacteroides fragilis NCTC 9434; Capnocytophaga ochracea DSM 7271; Flavobacterium psychrophilum JIP02 86; Akkermansia muciniphila ATCC BAA 835; Roseiflexus castenholzii DSM 13941; Roseiflexus RS1; Synechocystis PCC6803; Elusimicrobium minutum Pei191; uncultured Termite group 1 bacterium phylotype Rs D17; Fibrobacter succinogenes S85; Bacillus cereus ATCC 10987; Listeria innocua; Lactobacillus casei; Lactobacillus rhamnosus GG; Lactobacillus salivarius UCC 18; Streptococcus agalactiae A909; Streptococcus agalactiae NEM316; Streptococcus agalactiae 2603; Streptococcus dysgalactiae equisimilis GGS 124; Streptococcus equi zooepidemicus MGCS10565; Streptococcus gallolyticus UCN34 uid46061; Streptococcus gordonii Challis subst CH1; Streptococcus mutans NN2025 uid46353; Streptococcus mutans; Streptococcus pyogenes M1 GAS; Streptococcus pyogenes MGAS5005; Streptococcus pyogenes MGAS2096; Streptococcus pyogenes MGAS9429; Streptococcus pyogenes MGAS10270; Streptococcus pyogenes MGAS6180; Streptococcus pyogenes MGAS315; Streptococcus pyogenes SSI-1; Streptococcus pyogenes MGAS10750; Streptococcus pyogenes NZ131; Streptococcus thermophiles CNRZ1066; Streptococcus thermophiles LMD-9; Streptococcus thermophiles LMG 18311; Clostridium botulinum A3 Loch Marec; Clostridium botulinum B Eklund 17B; Clostridium botulinum Ba4 657; Clostridium botulinum F Langeland; Clostridium cellulolyticum H10; Finegoldia magna ATCC 29328; Eubacterium rectale ATCC 33656; Mycoplasma gallisepticum; Mycoplasma mobile 163K; Mycoplasma penetrans; Mycoplasma synoviae 53; Streptobacillus moniliformis DSM 12112; Bradyrhizobium BTAi1; Nitrobacter hamburgensis X14; Rhodopseudomonas palustris BisB18; Rhodopseudomonas palustris BisB5; Parvibaculum lavamentivorans DS-1; Dinoroseobacter shibae DFL 12; Gluconacetobacter diazotrophicus Pal 5 FAPERJ; Gluconacetobacter diazotrophicus Pal 5 JGI; Azospirillum B510 uid46085; Rhodospirillum rubrum ATCC 11170; Diaphorobacter TPSY uid29975, Verminephrobacter eiseniae EF01-2; Neisseria meningitides 053442; Neisseria meningitides alpha14; Neisseria meningitides Z2491; Desulfovibrio salexigens DSM 2638; Campylobacter jejuni doylei 269 97; Campylobacter jejuni 81116; Campylobacter jejuni; Campylobacter lari RM2100; Helicobacter hepaticus; Wolinella succinogenes; Tolumonas auensis DSM 9187; Pseudoalteromonas atlantica T6c; Shewanella pealeana ATCC 700345; Legionella pneumophila Paris; Actinobacillus succinogenes 130Z; Pasteurella multocida; Francisella tularensis novicida U112; Francisella tularensis holarctica; Francisella tularensis FSC 198; Francisella tularensis tularensis; Francisella tularensis WY96-3418; and Treponema denticola ATCC 35405. The Cas9 protein may be referred by one of skill in the art in the literature as Csn1. An exemplary S. pyogenes Cas9 protein sequence is shown below. See Deltcheva et al., Nature 471, 602-607 (2011) hereby incorporated by reference in its entirety.

MDKKYSIGLDIGTNSVGWAVITDEYKVTSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLYFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQDFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPE
DNEQKQLFVEQHKEYLDEIIEQISEFSKRVILADANLDKVLSAYNKHRDK
PIREQAENIIHLFTLTNLGAPAAFKYFDTTIDRKRYTSTKEVLDATLIHQ
SITGLYETRIDLSQLGGD

Modification to the Cas9 protein is a representative embodiment of the present disclosure. CRISPR systems useful in the present disclosure are described in R. Barrangou, P. Horvath, CRISPR: new horizons in phage resistance and strain identification. Annual review of food science and technology 3, 143 (2012) and B. Wiedenheft, S. H. Steinberg, J. A. Doudna. RNA-guided genetic silencing systems in bacteria and archaea. Nature 482, 331 (Feb. 16, 2012) each of which are hereby incorporated by reference in their entireties.

According to one aspect, a Cas9 protein having two or more nuclease domains may be modified or altered to inactivate all but one of the nuclease domains. Such a modified or altered Cas9 protein is referred to as a nickase, to the extent that the nickase cuts or nicks only one strand of double stranded DNA. According to one aspect, the Cas9 protein or Cas9 protein nickase includes homologs and orthologs thereof which retain the ability of the protein to bind to the DNA and be guided by the RNA. According to one aspect, the Cas9 protein includes the sequence as known for naturally occurring Cas9 proteins, such as that from S. pyogenes, S. thermophilus or S. aureus and protein sequences having at least 30%, 40%, 50%, 60%, 70%, 80%, 90%, 95%, 98% or 99% homology thereto and being a DNA binding protein, such as an RNA guided DNA binding protein.

Target nucleic acids include any nucleic acid sequence to which a co-localization complex as described herein can be useful to either cut, nick, regulate, identify, influence or otherwise target for other useful purposes using the methods described herein. Target nucleic acids include cellular RNA. Target nucleic acids include cellular DNA. Target nucleic acids include genes. For purposes of the present disclosure, DNA, such as double stranded DNA, can include the target nucleic acid and a co-localization complex can bind to or otherwise co-localize with the DNA at or adjacent or near the target nucleic acid and in a manner in which the co-localization complex may have a desired effect on the target nucleic acid. Such target nucleic acids can include endogenous (or naturally occurring) nucleic acids and exogenous (or foreign) nucleic acids. Target nucleic acids include DNA that encodes the modified guide RNA. One of skill based on the present disclosure will readily be able to identify or design guide RNAs and Cas9 proteins which co-localize to a DNA including a target nucleic acid. DNA includes genomic DNA, mitochondrial DNA, viral DNA or exogenous DNA.

Foreign nucleic acids (i.e. those which are not part of a cell's natural nucleic acid composition) may be introduced into a cell using any method known to those skilled in the art for such introduction. Such methods include transfection, transduction, viral transduction, microinjection, lipofection, nucleofection, nanoparticle bombardment, transformation, conjugation and the like. One of skill in the art will readily understand and adapt such methods using readily identifiable literature sources.

Vectors are contemplated for use with the methods and constructs described herein. The term “vector” includes a nucleic acid molecule capable of transporting another nucleic acid to which it has been linked. Vectors used to deliver the nucleic acids to cells as described herein include vectors known to those of skill in the art and used for such purposes. Certain exemplary vectors may be plasmids, lentiviruses or adeno-associated viruses known to those of skill in the art. Vectors include, but are not limited to, nucleic acid molecules that are single-stranded, doublestranded, or partially double-stranded; nucleic acid molecules that comprise one or more free ends, no free ends (e.g. circular); nucleic acid molecules that comprise DNA, RNA, or both, and other varieties of polynucleotides known in the art. One type of vector is a “plasmid.” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g. retroviruses, lentiviruses, replication defective retroviruses, adenoviruses, replication defective adenoviruses, and adeno-associated viruses). Viral vectors also include polynucleotides carried by a virus for transfection into a host cell. Certain vectors are capable of autonomous replication in a host cell into which they are introduced (e.g. bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” Common expression vectors of utility in recombinant DNA techniques are often in the form of plasmids. Recombinant expression vectors can comprise a nucleic acid of the invention in a form suitable for expression of the nucleic acid in a host cell, which means that the recombinant expression vectors include one or more regulatory elements, which may be selected on the basis of the host cells to be used for expression, that is operatively-linked to the nucleic acid sequence to be expressed. Within a recombinant expression vector, “operably linked” or “operatively linked” is intended to mean that the nucleotide sequence of interest is linked to the regulatory element(s) in a manner that allows for expression of the nucleotide sequence (e.g. in an in vitro transcription/translation system or in a host cell when the vector is introduced into the host cell).

Methods of non-viral delivery of nucleic acids or native DNA binding protein, native guide RNA or other native species include lipofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Felgner, WO 91/17424; WO 91/16024. Delivery can be to cells (e.g. in vitro or ex vivo administration) or target tissues (e.g. in vivo administration). The term native includes the protein, enzyme or guide RNA species itself and not the nucleic acid encoding the species.

Regulatory elements are contemplated for use with the methods and constructs described herein. The term “regulatory element” is intended to include promoters, enhancers, internal ribosomal entry sites (IRES), and other expression control elements (e.g. transcription termination signals, such as polyadenylation signals and poly-U sequences). Such regulatory elements are described, for example, in Goeddel, GENE EXPRESSION TECHNOLOGY: METHODS IN ENZYMOLOGY 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in a desired tissue of interest, such as muscle, neuron, bone, skin, blood, specific organs (e.g. liver, pancreas), or particular cell types (e.g. lymphocytes). Regulatory elements may also direct expression in a temporal-dependent manner, such as in a cell-cycle dependent or developmental stage-dependent manner, which may or may not also be tissue or cell-type specific. In some embodiments, a vector may comprise one or more pol III promoter (e.g. 1, 2, 3, 4, 5, or more pol III promoters), one or more pol II promoters (e.g. 1, 2, 3, 4, 5, or more pol II promoters), one or more pol 1 promoters (e.g. 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) [see, e.g., Boshart et al, Cell, 41:521-530 (1985)], the SV40 promoter, the dihydrofolate reductase promoter, the β-actin promoter, the phosphoglycerol kinase (PGK) promoter, and the EF1α promoter and Pol II promoters described herein. Also encompassed by the term “regulatory element” are enhancer elements, such as WPRE; CMV enhancers; the R-U5′ segment in LTR of HTLV-I (Mol. Cell. Biol., Vol. 8(1), p. 466-472, 1988); SV40 enhancer; and the intron sequence between exons 2 and 3 of rabbit β-globin (Proc. Natl. Acad. Sci. USA., Vol. 78(3), p. 1527-31, 1981). It will be appreciated by those skilled in the art that the design of the expression vector can depend on such factors as the choice of the host cell to be transformed, the level of expression desired, etc. A vector can be introduced into host cells to thereby produce transcripts, proteins, or peptides, including fusion proteins or peptides, encoded by nucleic acids as described herein (e.g., clustered regularly interspersed short palindromic repeats (CRISPR) transcripts, proteins, enzymes, mutant forms thereof, fusion proteins thereof, etc.).

Aspects of the methods described herein may make use of terminator sequences. A terminator sequence includes a section of nucleic acid sequence that marks the end of a gene or operon in genomic DNA during transcription. This sequence mediates transcriptional termination by providing signals in the newly synthesized mRNA that trigger processes which release the mRNA from the transcriptional complex. These processes include the direct interaction of the mRNA secondary structure with the complex and/or the indirect activities of recruited termination factors. Release of the transcriptional complex frees RNA polymerase and related transcriptional machinery to begin transcription of new mRNAs. Terminator sequences include those known in the art and identified and described herein.

Aspects of the methods described herein may make use of epitope tags and reporter gene sequences. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-S-transferase (GST), horseradish peroxidase (HRP), chloramphenicol acetyltransferase (CAT) beta-galactosidase, betaglucuronidase, luciferase, green fluorescent protein (GFP), HcRed, DsRed, cyan fluorescent protein (CFP), yellow fluorescent protein (YFP), and autofluorescent proteins including blue fluorescent protein (BFP).

The following examples are set forth as being representative of the present disclosure. These examples are not to be construed as limiting the scope of the present disclosure as these and other equivalent embodiments will be apparent in view of the present disclosure, figures and accompanying claims.

### Examples

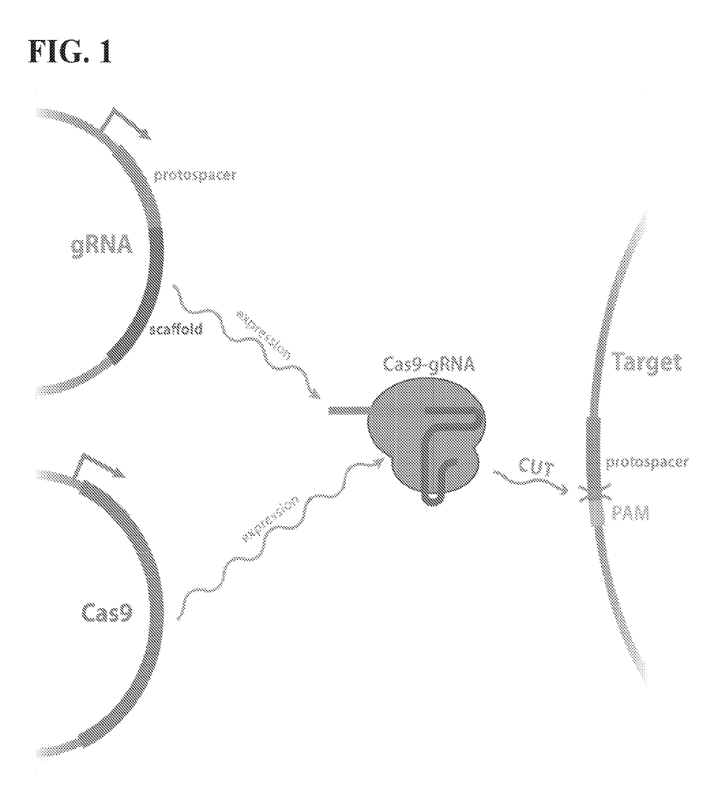

In standard applications of the CRISPR/Cas9 system, the Cas9 protein and guide RNAs are introduced into the cells by one or multiple DNA vectors. The cells express the Cas9 protein and the gRNA. The Cas9 protein and gRNA combine and form a co-localization complex at the target nucleic acid loci that contain both the matching protospacer sequence and the PAM where the Cas9 cuts the endogenous target nucleic acid loci (FIG. 1). The Cas9-gRNA complex does not attack the DNA vector containing the gRNA gene because the DNA vector that encodes the gRNA gene while containing a cognate protospacer sequence. It does not contain a PAM sequence adjacent to the protospacer sequence.

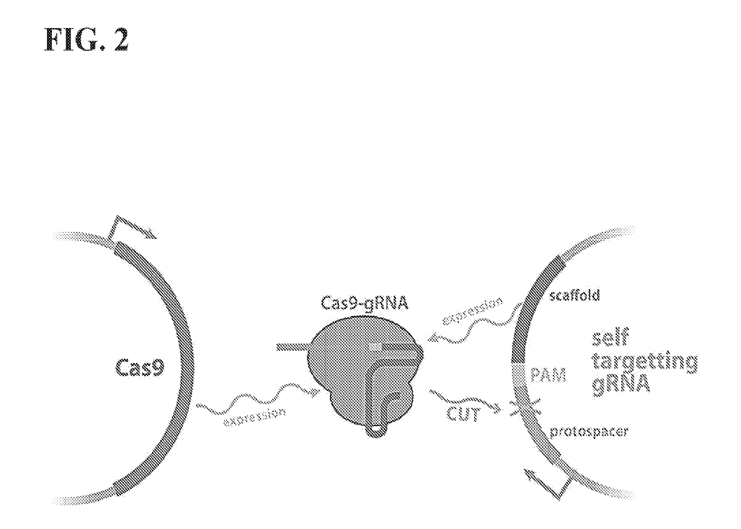

There are applications where targeting a gRNA locus by its own gRNA product can be desirable. In this example, modified Cas9 gRNA scaffolds are designed and used to cut a gRNA locus (DNA encoding the gRNA) by its own gRNA product, i.e., a self-targeting gRNA (FIG. 2).

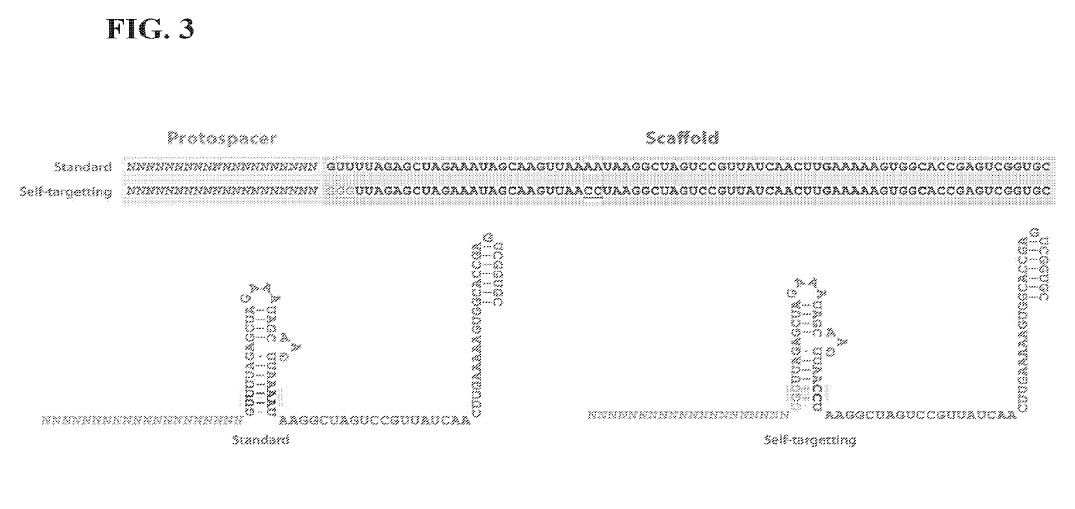

In an exemplary embodiment, a Streptococcus pyogenes gRNA sequence was modified to introduce a PAM adjacent to the spacer sequence while minimally altering the secondary structure of the gRNA scaffold (FIG. 3). These novel gRNAs were tested in standard traffic-light assays (e.g., as described in Certo M T, Ryu B Y, Annis J E, Garibov M. Jarjour J, Rawlings D J, Scharenberg A M., Tracking genome engineering outcome at individual DNA breakpoints, Nat Methods., 2011 Jul. 10; 8(8):671-6, hereby incorporated by reference in its entirety) and appeared to be active in targeting both a genomic locus and the encoding DNA vector itself, indicating that these gRNAs can be used for genome engineering applications, as well as eliminating themselves during the process that leaves no active genetic elements behind.

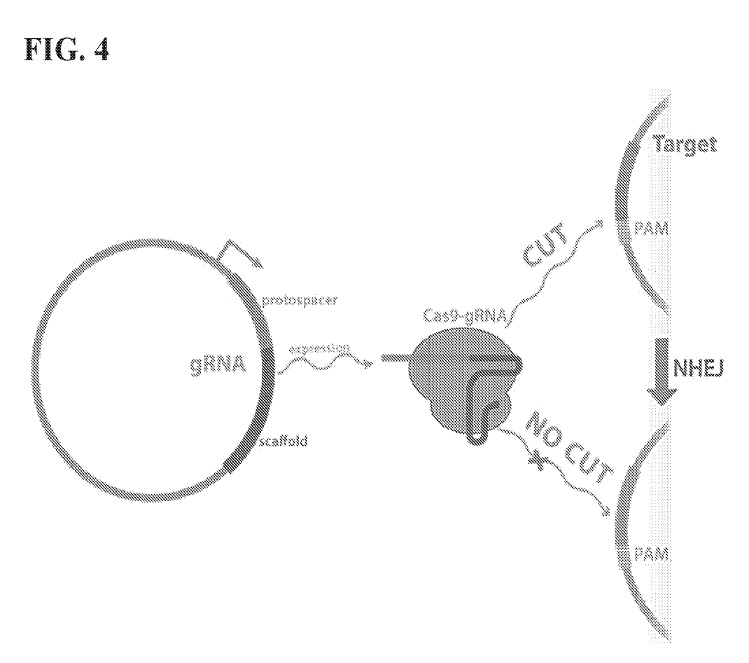

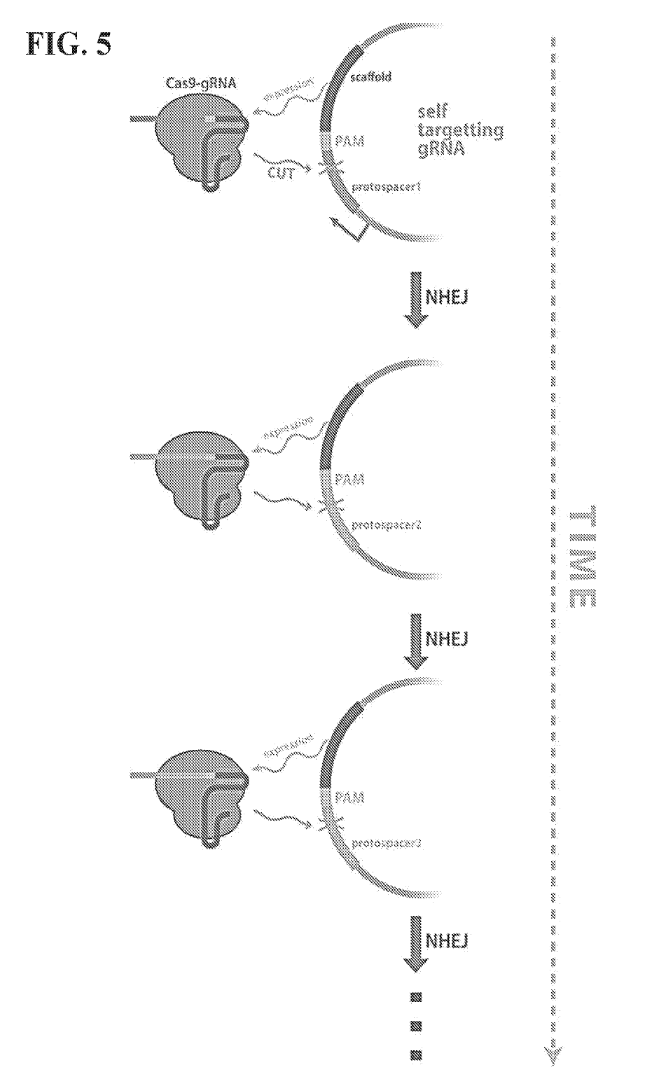

Standard gRNAs can only create one alteration in their target nucleic acid sequence in cells because the spacer sequences of the standard gRNAs do not match their target nucleic acid protospacer sequences anymore after the target nucleic acid protospacer sequences have been altered, such as by NHEJ repair (FIG. 4). In contrast, a modified self-targeting gRNA according to an embodiment of the invention can repeatedly attack its own DNA encoding locus because after each round of alteration, the altered DNA encoding locus that is repaired by NHEJ will express a gRNA that carries the newly altered spacer sequence that matches the altered protospacer sequence of the gRNA locus (FIG. 5).

The CRISPR Cas9 Self-Targeting gRNA System Targets the gRNA Encoding Locus in Cells

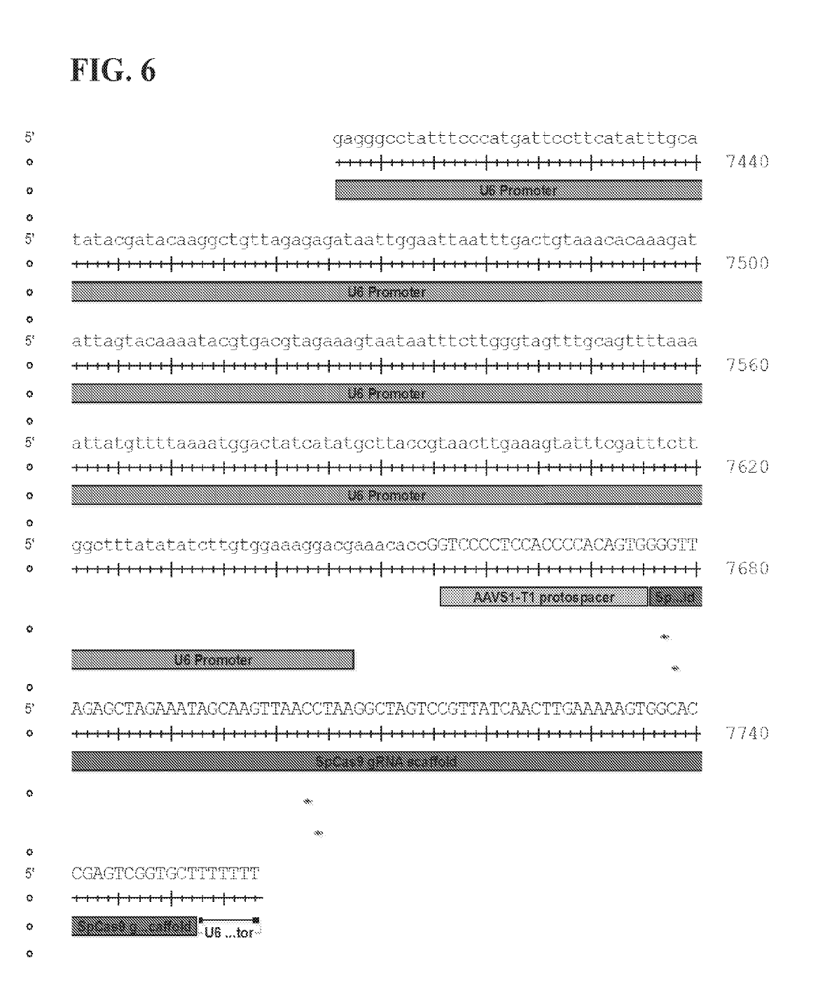

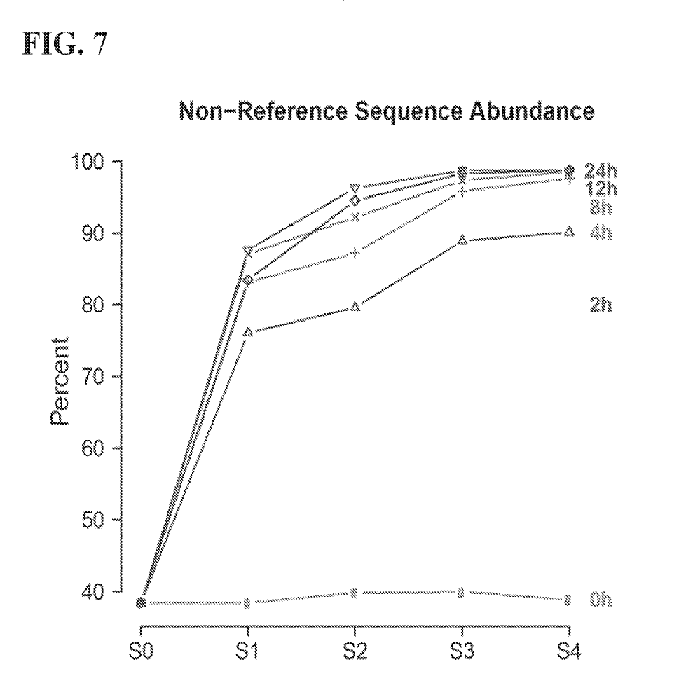

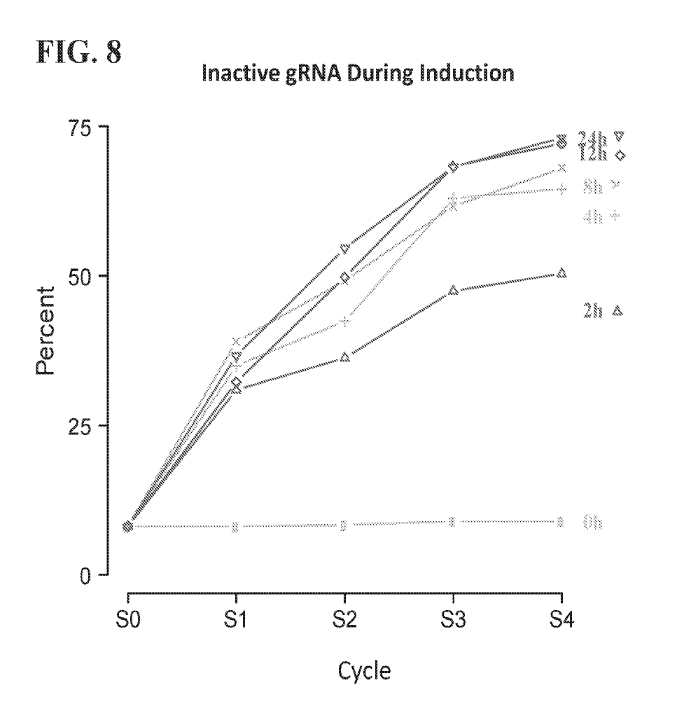

The behavior of a self-targeting gRNA (FIG. 3 and FIG. 6) was tested by introducing it to cells with an inducible Cas9 protein. Cas9 expression was then induced for pulses of 0, 2, 4, 8, 12, or 24 hours within 72 hour intervals. The gRNA locus was sequenced at the end of each interval (FIG. 7). The initial or input gRNA locus sequence is designated as the reference sequence. The gRNA locus sequence/read that have been altered by the self-targeting gRNA/Cas9 complex and repaired by NHEJ are designated as non-reference sequence. The results suggested that the gRNA locus changed over time with increased abundance of the non-reference sequence corresponds to the increased Cas9 protein induction time. After a few rounds of induction of Cas9 protein, an NHEJ event involving a large deletion that removes the PAM sequence from the gRNA locus eventually happened, thus rendering the gRNA locus non-functional/inactive as a target (FIG. 8). This outcome indicated that the CRISPR Cas9 self-targeting gRNA system may be used as molecular clocks or for measuring or recording various cellular events including, but are not limited to, divisions, lineage, and signaling that can be coupled to Cas9 expression. This system can have additional applications related to cellular and molecular barcoding, lineage tracing, measurement and recording of various cellular signals that can be coupled to the production of Cas9 protein or gRNA, and creating suicidal gRNAs that inactivate or eliminate themselves after a certain amount of time.

### Materials and Methods

A clonal HeLa cell line with a genomically integrated, doxycycline-inducible, SP-Cas9 was obtained (HeLa-iSPCas9 cells).

A self-targeting guide RNA gene under U6 promoter (FIG. 6) was cloned into a lentiviral vector backbone with Hygromycin resistance gene as a selectable marker (stgRNA1) using standard technique known to a skilled in the art (See, e.g., Lois C, Hong E J, Pease S, Brown E J, Baltimore D., Germline transmission and tissue-specific expression of transgenes delivered by lentiviral vectors, Science, 2002, Feb. 1; 295(5556):868-72, Epub 2002 Jan. 10, PubMed PMID: 11786607, hereby incorporated by reference in its entirety).

A lentiviral virus library carrying this self-targeting guide RNA gene vector were produced into HEK/293T cells (stgRNA1 lentiviral library).

HeLa-iSPCas9 cells were transduced with the stgRNA1 lentiviral library in the presence of 6 microgram/ml polybrene. Two days after transduction, cells were placed under 200 microgram/ml Hygromycin selection and passaged for one week under selection to eliminate the cells that were not transduced with the lentiviral virus, resulting in a cell culture of HeLa-iSPCas9-stgRNA1.

The HeLa-iSPCas9-stgRNA1 cells were passaged into a 6-well culture dish. A sample of the uninduced cells was taken and their genomic DNAs were extracted (50 sample).

After the cells attached to the bottom of the 6-well culture dish, cells in wells 1 through 6 were respectively induced for 0, 2, 4, 8, 12, and 24 hours with 2 μg/ml doxycycline (Dox) to induce SP-Cas9 expression. At the end of each induction time, the cells of the corresponding well was washed twice with fresh culture medium and cultured in Dox-free medium. The 0 hour-induced sample was used as a no-induction negative control.

Three days after induction, 90% of the cells in each well were harvested and their genomic DNAs were extracted (S1 samples). The remaining 10% of the cells were passaged into a new 6-well culture dish and induced once again as previously done, with each well receiving the same amount of induction time (i.e., 0, 2, 4, 8, 12, or 24 hours) as their respective parent well had received. Again, three days after induction, 90% of the cells in each well were harvested and their DNAs were extracted (S2 samples). These induction and DNA extraction steps were repeated two more times to obtain S3 and S4 samples, with each sample having 6 induction time lengths.

The genomic DNAs from all obtained samples were extracted using Qiagen DNAeasy Blood and Tissue Kit. The table 1 below lists the time and rounds of all the samples obtained:

TABLE 1

| | | Induction Round | | | | |
|---|---|---|---|---|---|---|
| | | 0 | 1 | 2 | 3 | 4 |
| Induction | 0 hours | S0 | 0 h-S1 | 0 h-S2 | 0 h-S3 | 0 h-S4 |
| Length | 1 hour | | 1 h-S1 | 1 h-S2 | 1 h-S3 | 1 h-S4 |
| | 2 hours | | 2 h-S1 | 2 h-S2 | 2 h-S3 | 2 h-S4 |
| | 4 hours | | 4 h-S1 | 4 h-S2 | 4 h-S3 | 4 h-S4 |
| | 8 hours | | 8 h-S1 | 8 h-S2 | 8 h-S3 | 8 h-S4 |
| | 12 hours | | 12 h-S1 | 12 h-S2 | 12 h-S3 | 12 h-S4 |
| | 24 hours | | 24 h-S1 | 24 h-S2 | 24 h-S3 | 24 h-S4 |

For each extracted DNA sample, the stgRNA locus was amplified in a first round of PCR amplification with the following primers:

Forward primer:
atggactatcatatgcttaccgt
Reverse primer:
TTCAAGTTGATAACGGACTAGC

PCR was done with an initial denaturation of 5 minutes at 95° C. 25 cycles at 95° C. for 30 seconds and at 65° C. for 1 minute, with a final extension of 5 minutes at 72° C.

In a second round of PCR amplification, the PCR product from the first round was amplified with NEBNext Indexing Sets 1 and 2. The now-indexed products of this second PCR amplification round were combined into a library for subsequent DNA sequencing. This library was sequenced using Illumina MiSeq platform with 150 bp single-end reads and 8 bp index reads.

Evaluation of sequencing results clearly revealed the self-targeting behavior of these guide RNAs (FIG. 7). Whereas before induction (50 sample), more than 75% of the sequenced stgRNAs match the exact sequence of stgRNA1 in FIG. 6. With each induction round and corresponding with induction time length, the stgRNA sequences started changing as the non-homologous end joining repair (NHEJ) repairs the cuts the self-targeting gRNAs have introduced upon their target loci while introducing sequence alterations (non-reference sequence). Eventually, in the 24 hour-induced sample and after four rounds of induction, less than 2% of all sequenced guide RNAs have their original sequence (reference sequence) as in FIG. 6. The type of sequence alterations that are produced involved mostly deletions which are similar to alterations that are known to be a result of NHEJ repair.

From the sequencing results, it was also observed that, after multiple rounds of induction, the stgRNA locus underwent multiple cycles of cutting and repairing, the stgRNA locus eventually became inactive as the NHEJ repair process eventually led to a large deletion that encompasses the PAM and/or the gRNA scaffold (FIG. 8).

The Rate of Changing the gRNA Locus can be Regulated by the Length of the Self-Targeting gRNA

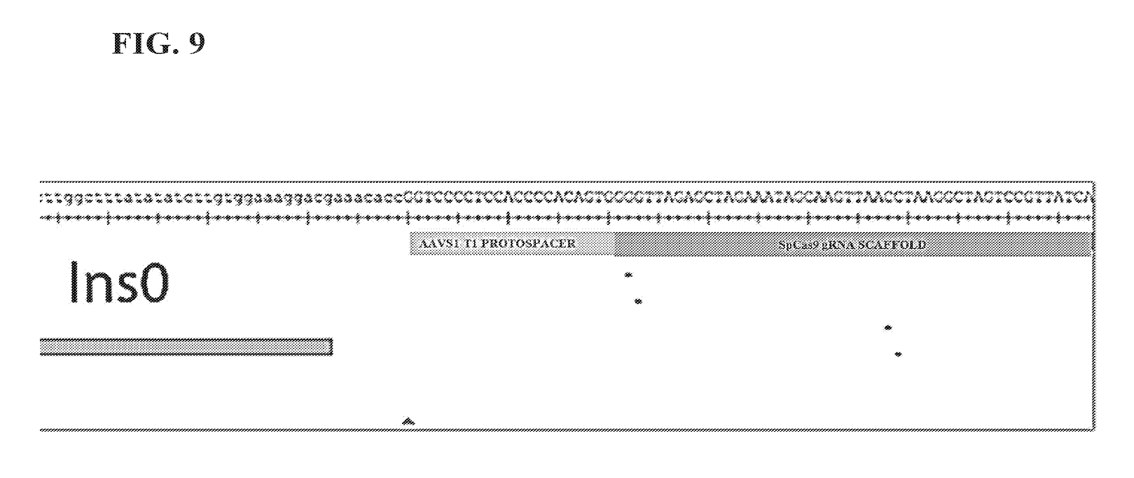

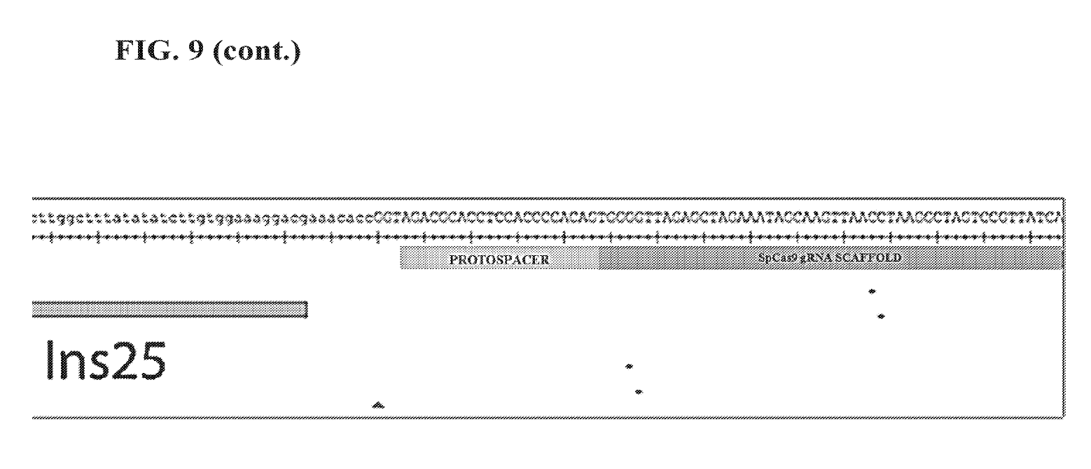

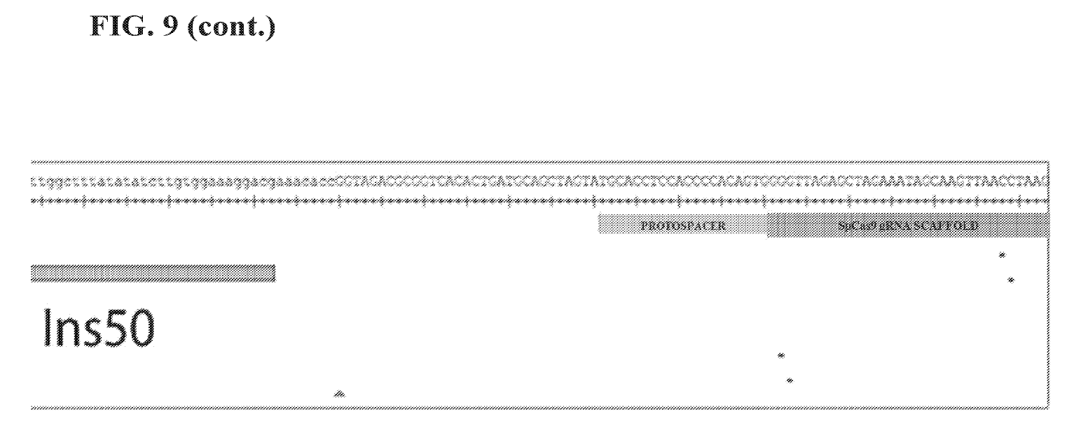

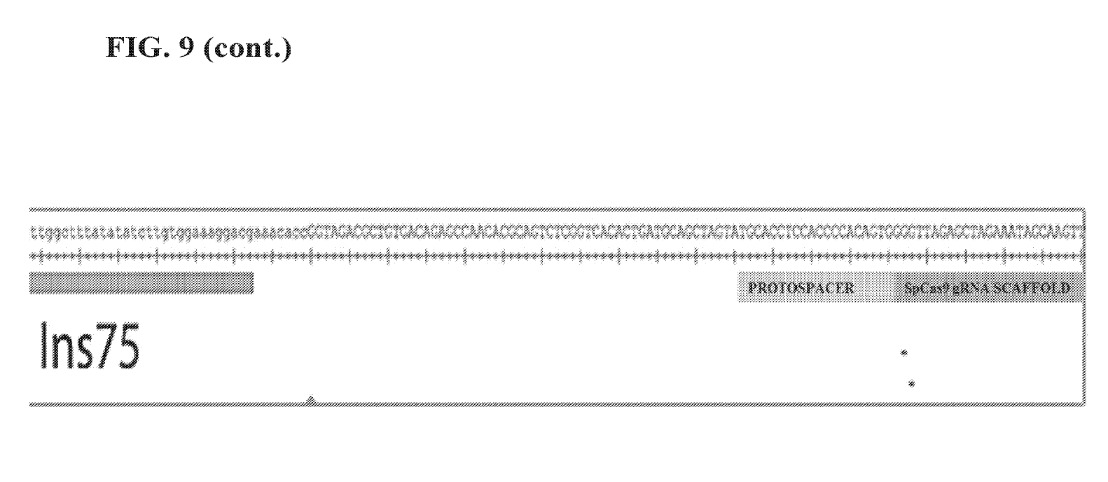

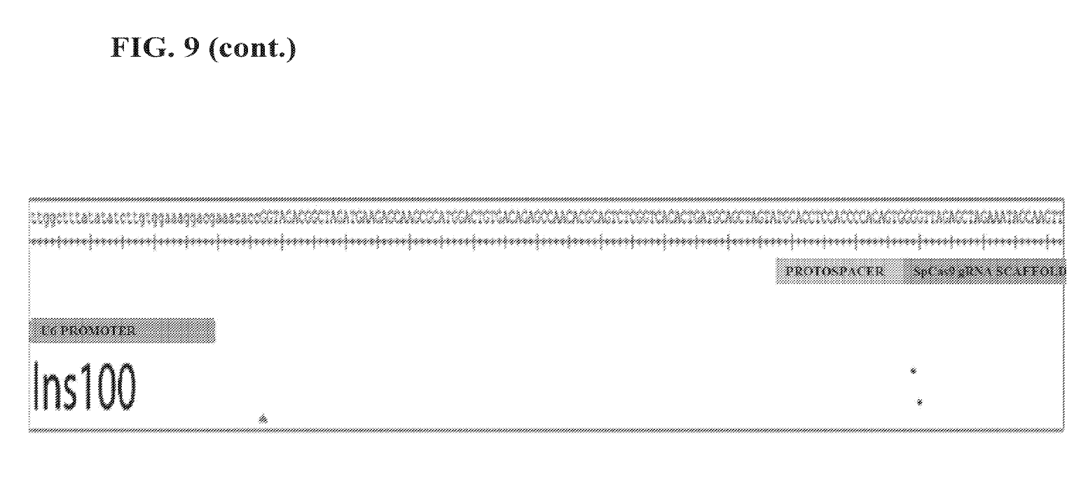

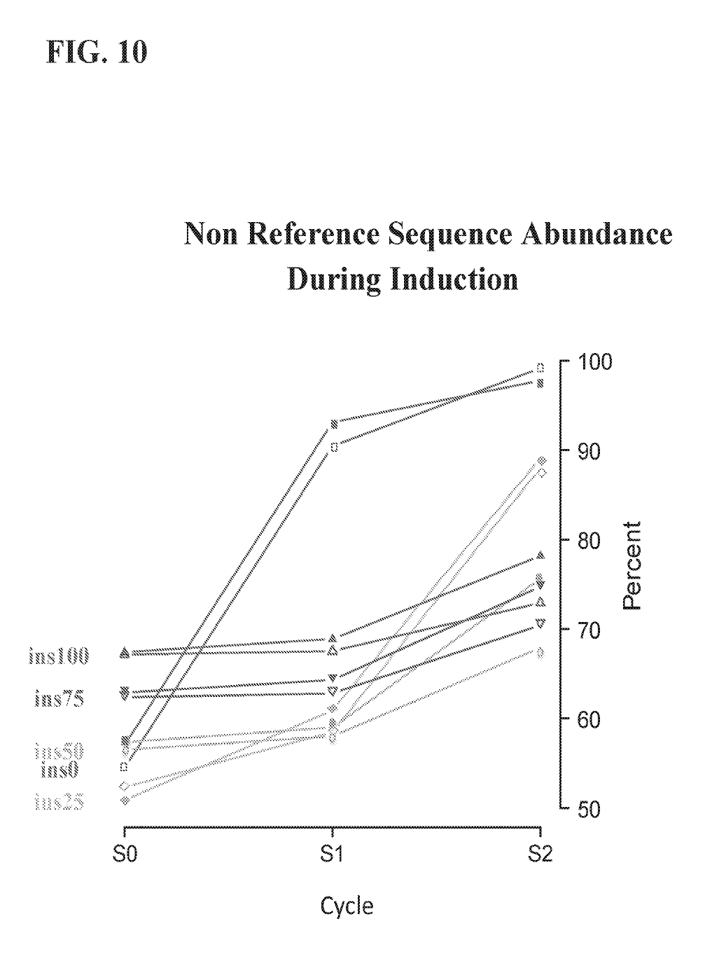

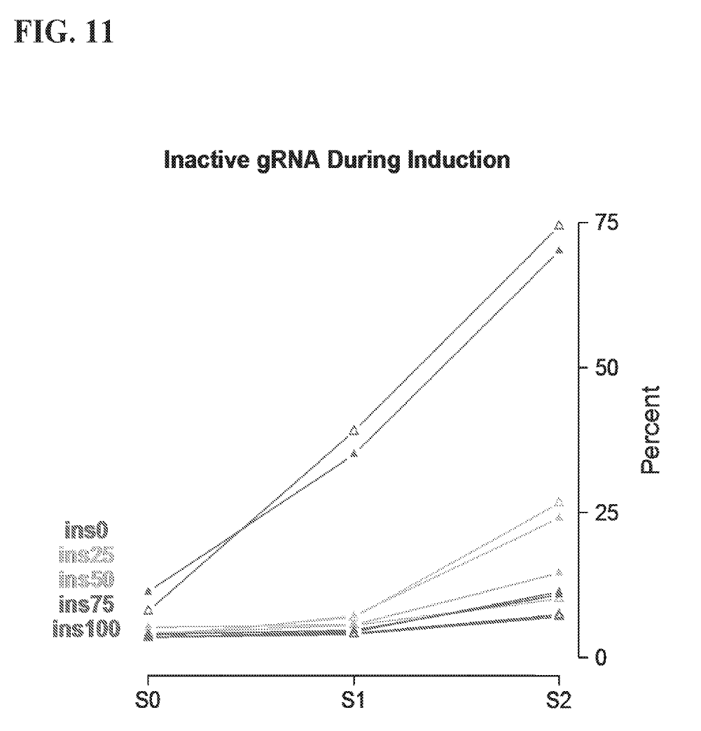

To find out if the rate of changing/altering the gRNA locus by the self-targeting gRNA for such a molecular-clock can be controlled, four additional self-targeting gRNAs were created, each with an additional 25 bases of nucleotide sequence added to its 5′ end, between the transcription start site and the gRNA scaffold (FIG. 9). The additional nucleotide sequence likely reduces the expression level of the gRNA and subsequently affects the rate at which the expressed gRNA changes/alters the sequence of the gRNA locus. Each of these gRNAs was introduced to a Cas9 expressing cell line, the cells were induce and sample DNAs were collected as previously described herein. Sequencing results from these samples clearly revealed the self-targeting behavior of these guide RNAs (FIG. 10). The fraction of gRNA loci that lost their PAM sequence and rendered inactive over time were measured (FIG. 11). The results indicated that the rate at which these gRNAs regulate/alter their own gRNA loci can be controlled, with increasing sequence length at the 5′ end of the gRNA between the transcription start site and the gRNA scaffold leading to reduced rate of sequence alterations.

### Materials and Methods

Five self-targeting guide RNA genes under U6 promoter were cloned into their respective lentiviral vector backbones with Hygromycin resistance gene as a selectable marker as previously described herein. Each gRNA gene has a different distance between its transcriptional start site and the gRNA scaffold (FIG. 9), with ins0 representing the shortest distance and ins100 representing the longest distance.

Lentiviral virus libraries carrying each of these self-targeting guide RNA genes were produced in HEK/293T cells (ins0-stgRNA, ins25-stgRNA, ins50-stgRNA, ins75-stgRNA, and ins100-stgRNA lentiviral libraries).

HeLa-iSPCas9 cells were transduced with each of the ins0-stgRNA, ins25-stgRNA, ins50-stgRNA, ins75-stgRNA, and ins100-stgRNA expressing lentiviral libraries in the presence of 6 microgram/ml polybrene. Two days after transduction, cells were placed under Hygromycin selection and passaged in cell culture dishes for one week under selection to eliminate the cells that were not transduced with the lentiviruses, producing HeLa-iSPCas9-ins0. HeLa-iSPCas9-ins25, HeLa-iSPCas9-ins50, HeLa-iSPCas9-ins75, and HeLa-iSPCas9-ins100 cell lines.

Genomic DNAs of a sample of un-induced cells from each of the cell lines (HeLa-iSPCas9-ins0, HeLa-iSPCas9-ins25, HeLa-iSPCas9-ins50, HeLa-iSPCas9-ins75, and HeLa-iSPCas9-ins100) were extracted to obtain the corresponding non-induced 50 samples as previously described herein.

The cells of each cell line were then passaged into a new cell culture dish and induced for 48 hours with 2 μg/ml Doxycycline (Dox) to induce SP-Cas9 expression. At the end of induction, all samples were washed twice with fresh culture medium and cultured in Dox-free medium.

Three days after induction, 90% of the cells in the culture dishes of each cell line were harvested and their genomic DNAs were extracted (S1 samples). The remaining 10% of the cells in the culture dishes of each cell line were passaged into new cell culture dishes and induced once again as described in the previous step herein. Three days after induction, 90% of the cells in the culture dishes of each cell line were harvested and their genomic DNAs were extracted (S2 samples).

The genomic DNAs from all obtained samples were extracted using Qiagen DNAeasy Blood and Tissue Kit. The table 2 below lists the time and rounds of all the samples obtained:

TABLE 2

| | | Induction Round | | |
|---|---|---|---|---|
| | | 0 | 1 | 2 |
| stgRNA | ins0 | ins0-S0 | ins0-S1 | ins0-S2 |
| | ins25 | ins25-S0 | ins25-S1 | ins25-S2 |
| | ins50 | ins50-S0 | ins50-S1 | ins50-S2 |
| | ins75 | ins75-S0 | ins75-S1 | ins75-S2 |
| | ins100 | ins100-S0 | ins100-S1 | ins100-S2 |

For each extracted DNA sample, the stgRNA locus was amplified in a first round of PCR amplification with the following primers as previously described herein:

Forward primer:
atggactatcatatgcttaccgt
Reverse primer:
TTCAAGTTGATAACGGACTAGC

In a second round of PCR amplification, the PCR product from the first round was amplified with NEBNext Indexing Sets 1 and 2. The now-indexed products of this second PCR amplification round were combined into a library for subsequent DNA sequencing. This library was sequenced using Illumina MiSeq platform with 200 bp single-end reads and 8 bp index reads.

Evaluation of the sequencing results clearly revealed the self-targeting behavior for each of the five stgRNAs (FIG. 10). It was observed that the self-targeting efficiency varied among samples and can be reduced by increasing the distance between the gRNA transcription start site and the gRNA scaffold in the gRNA locus (FIG. 10 and FIG. 11).

What is claimed is:

1. A method of targeting a nucleic acid encoding a guide RNA in a cell comprising
introducing into the cell a first foreign nucleic acid encoding a guide RNA sequence including a spacer sequence and a protospacer adjacent motif (PAM) adjacent to the spacer sequence, wherein the spacer sequence is complementary to a protospacer sequence in the first foreign nucleic acid and to a protospacer sequence in a target nucleic acid sequence of the genomic DNA,
introducing into the cell a second foreign nucleic acid encoding a Cas9 protein,
wherein the guide RNA sequence and the Cas9 protein are expressed, and
wherein the guide RNA sequence and the Cas9 protein co-localize to the first foreign nucleic acid and the Cas9 protein binds or cleaves the first foreign nucleic acid sequence in a site specific manner.

2. The method of claim 1, wherein the binding or cleaving of the first foreign nucleic acid sequence alters the expression of the guide RNA or inactivates the first foreign nucleic acid sequence encoding the guide RNA.

3. The method of claim 1, wherein the guide RNA and the Cas9 protein co-localize to the target nucleic acid sequence and the Cas9 protein binds or cleaves the target nucleic acid sequence in a site specific manner.

4. The method of claim 3, wherein the binding or cleaving of the target nucleic acid sequence alters the expression of the target nucleic acid sequence.

5. The method of claim 1, wherein the first foreign nucleic acid sequence that is cleaved in a site specific manner is repaired by non-homologous end joining repair mechanism to form a repaired subsequent foreign nucleic acid sequence encoding a subsequent guide RNA having a subsequent spacer sequence complementary to a subsequent target nucleic acid sequence of the genomic DNA.

6. The method of claim 5, wherein the repaired subsequent foreign nucleic acid sequence is expressed to form the subsequent guide RNA which forms a colocalization complex with the Cas9 protein and the repaired subsequent foreign nucleic acid sequence, wherein the Cas9 protein cleaves the repaired subsequent foreign nucleic acid sequence in a site specific manner to prevent further expression of the subsequent guide RNA sequence.

7. The method of claim 5, wherein the subsequent guide RNA and the Cas9 protein co-localize to the subsequent target nucleic acid sequence and the Cas9 protein cleaves the subsequent target nucleic acid sequence in a site specific manner.

8. The method of claim 5, wherein the process of cleaving the first foreign nucleic acid sequence, repairing the first foreign nucleic acid sequence, expressing the repaired subsequent foreign nucleic acid sequence, cleaving the repaired subsequent foreign nucleic acid sequence in a site specific manner, and cleaving the subsequent target nucleic acid sequence in a site specific manner is cycled in the cell to result in (1) eliminating or inactivating the foreign nucleic acid sequence and (2) a plurality of target nucleic acid sequences being cleaved.

9. The method of claim 1, wherein the Cas9 is a Type II CRISPR system Cas9 or Cpf1.

10. The method of claim 1, wherein the Cas9 protein is an enzymatically active Cas9 protein, a Cas9 protein nickase, or a nuclease null Cas9 protein.

11. The method of claim 10, wherein the Cas9 protein further comprises a transcriptional regulator or a DNA modifying protein attached thereto.

12. The method of claim 1, wherein the cell is a eukaryotic cell or prokaryotic cell.

13. The method of claim 1, wherein the cell is a bacteria cell, yeast cell, a mammalian cell, a human cell, a plant cell or an animal cell.

14. The method of claim 1, wherein the rate at which the guide RNA regulates the binding or cleavage of the first foreign nucleic acid sequence and/or the target nucleic acid sequence can be controlled by adding additional nucleotide sequence between the transcription start site and the scaffold of the guide RNA.

15. The method of claim 14, wherein increasing the length of the additional nucleotide sequence between the transcription start site and the scaffold of the guide RNA reduces the rate at which the guide RNA regulates the binding or cleavage of the first foreign nucleic acid sequence and/or the target nucleic acid sequence.

16. The method of claim 1, wherein the method can be used for cellular and molecular barcoding.

17. The method of claim 1, wherein the method can be used to measure and record various cellular events that are coupled to production of the Cas9 protein or the guide RNA.

18. The method of claim 17, wherein the cellular events include cell divisions, lineage tracing and cellular signaling.

19. The method of claim 1, wherein the first and/or the second foreign nucleic acid sequence are exogenous to the cell.

20. The method of claim 1, wherein the first and/or the second foreign nucleic acid sequence are integrated into the cell's genomic DNA.

21. The method of claim 1, wherein the expression of the Cas9 protein is inducible.

22. The method of claim 1, wherein the Cas9 protein is introduced.

23. A method of targeting a nucleic acid encoding a guide RNA in vitro comprising
providing a first foreign nucleic acid encoding a guide RNA sequence including a spacer sequence and a protospacer adjacent motif (PAM) adjacent to the spacer sequence, wherein the spacer sequence is complementary to a protospacer sequence in the first foreign nucleic acid,
providing a second foreign nucleic acid encoding a Cas9 protein,
wherein the guide RNA sequence and the Cas9 protein are expressed, and
wherein the guide RNA sequence and the Cas9 protein co-localize to the first foreign nucleic acid and the Cas9 protein binds or cleaves the first foreign nucleic acid sequence in a site specific manner.

24. The method of claim 23, wherein the binding or cleaving of the first foreign nucleic acid sequence alters the expression of the guide RNA or inactivates the first foreign nucleic acid sequence encoding the guide RNA.

25. The method of claim 23, wherein other DNA having a target nucleic acid sequence is further provided, wherein the spacer sequence of the guide RNA is complementary to a protospacer sequence in the target nucleic acid sequence, and wherein the guide RNA and the Cas9 protein co-localize to the target nucleic acid sequence and the Cas9 protein binds or cleaves the target nucleic acid sequence in a site specific manner.

26. The method of claim 25, wherein the binding or cleaving of the target nucleic acid sequence alters the expression of the target nucleic acid sequence.

27. The method of claim 23, wherein the Cas9 is a Type II CRISPR system Cas9 or Cpf1.

28. The method of claim 23, wherein the Cas9 protein is an enzymatically active Cas9 protein, a Cas9 protein nickase, or a nuclease null Cas9 protein.

29. The method of claim 28, wherein the Cas9 protein further comprises a transcriptional regulator or a DNA modifying protein attached thereto.

30. The method of claim 23, wherein the guide RNA is provided.

31. The method of claim 23, wherein the Cas9 protein is provided.

32. The method of claim 25, wherein the rate at which the guide RNA regulates the binding or cleavage of the first foreign nucleic acid sequence and/or the target nucleic acid sequence can be controlled by adding additional nucleotide sequence between the transcription start site and the scaffold of the guide RNA.

33. The method of claim 32, wherein increasing the length of the additional nucleotide sequence between the transcription start site and the scaffold of the guide RNA reduces the rate at which the guide RNA regulates the binding or cleavage of the first foreign nucleic acid sequence and/or the target nucleic acid sequence.

34. The method of claim 23, wherein the method can be used for molecular cloning and genetic engineering applications.

35. The method of claim 23, wherein the method can be used to deplete or enrich specific targets in a library of DNA molecules.

36. The method of claim 23, wherein the first and/or the second foreign nucleic acid sequence are genomic DNA or exogenous to the genomic DNA.

37. The method of claim 23, wherein the first and/or the second foreign nucleic acid sequence are integrated into the genomic DNA.

38. The method of claim 23, wherein the activity or expression of the Cas9 protein is inducible.

39. A cell comprising
a first foreign nucleic acid encoding a guide RNA sequence including a spacer sequence and a protospacer adjacent motif (PAM) adjacent to the spacer sequence, wherein the spacer sequence is complementary to a protospacer sequence in the first foreign nucleic acid and a protospacer sequence in a target nucleic acid sequence of the genomic DNA,
a second foreign nucleic acid encoding a Cas9 protein,
wherein the guide RNA sequence and the Cas9 protein are expressed, and
wherein the guide RNA sequence and the Cas9 protein co-localize to the first foreign nucleic acid and the Cas9 protein binds or cleaves the first foreign nucleic acid sequence in a site specific manner.

40. The cell of claim 39, wherein the binding or cleaving of the first foreign nucleic acid sequence alters the expression of the guide RNA or inactivates the first foreign nucleic acid sequence encoding the guide RNA.

41. The cell of claim 39, wherein the guide RNA and the Cas9 protein co-localize to the target nucleic acid sequence and the Cas9 protein binds or cleaves the target nucleic acid sequence in a site specific manner.

42. The cell of claim 39, wherein the cell is a eukaryotic cell or prokaryotic cell.

43. The cell of claim 39, wherein the cell is a bacteria cell, yeast cell, a mammalian cell, a human cell, a plant cell or an animal cell.

44. The cell of claim 39, wherein the first and/or the second foreign nucleic acid sequence are exogenous to the cell.

45. The cell of claim 39, wherein the first and/or the second foreign nucleic acid sequence are integrated into the cell's genomic DNA.

46. The cell of claim 39, wherein the expression of the Cas9 protein is inducible.

47. An in vitro CRISPR system comprising
a first foreign nucleic acid encoding a guide RNA sequence including a spacer sequence and a protospacer adjacent motif (PAM) adjacent to the spacer sequence, wherein the spacer sequence is complementary to a protospacer sequence in the first foreign nucleic acid,
a second foreign nucleic acid encoding a Cas9 protein,
wherein the guide RNA sequence and the Cas9 protein are expressed, and
wherein the guide RNA sequence and the Cas9 protein co-localize to the first foreign nucleic acid and the Cas9 protein binds or cleaves the first foreign nucleic acid sequence in a site specific manner.

48. The in vitro CRISPR system of claim 47, wherein the binding or cleaving of the first foreign nucleic acid sequence alters the transcription of the guide RNA or inactivates the first foreign nucleic acid sequence encoding the guide RNA.

49. The in vitro CRISPR system of claim 47, further comprising a DNA library having a target nucleic acid sequence, wherein the spacer sequence of the guide RNA is complementary to a protospacer sequence in the target nucleic acid sequence, and wherein the guide RNA and the Cas9 protein co-localize to the target nucleic acid sequence and the Cas9 protein binds or cleaves the target nucleic acid sequence in a site specific manner.

50. The in vitro CRISPR system of claim 49, wherein the binding or cleaving of the target nucleic acid sequence alters the activity of the target nucleic acid sequence.

51. The in vitro CRISPR system of claim 47, wherein the Cas9 is a Type II CRISPR system Cas9 or Cpf1.

52. The in vitro CRISPR system of claim 47, wherein the Cas9 protein is an enzymatically active Cas9 protein, a Cas9 protein nickase, or a nuclease null Cas9 protein.

53. The in vitro CRISPR system of claim 52, wherein the Cas9 protein further comprises a transcriptional regulator or a DNA-modifying protein attached thereto.

54. The in vitro CRISPR system of claim 47, the guide RNA is provided.

55. The in vitro CRISPR system of claim 47, wherein the Cas9 protein is provided.

56. The in vitro CRISPR system of claim 49, wherein the rate at which the guide RNA regulates the binding or cleavage of the first foreign nucleic acid sequence and/or the target nucleic acid sequence can be controlled by adding additional nucleotide sequence between the transcription start site and the scaffold of the guide RNA.

57. The in vitro CRISPR system of claim 56, wherein increasing the length of the additional nucleotide sequence between the transcription start site and the scaffold of the guide RNA reduces the rate at which the guide RNA regulates the binding or cleavage of the first foreign nucleic acid sequence and/or the target nucleic acid sequence.

58. The in vitro CRISPR system of claim 47, wherein the first and/or the second foreign nucleic acid sequence are a library of DNA molecules.

59. The in vitro CRISPR system of claim 47, wherein the first and/or the second foreign nucleic acid sequence are integrated into the library of DNA molecules.

60. The in vitro CRISPR system of claim 47, wherein the activity or expression of the Cas9 protein is inducible.

61. A method of targeting a nucleic acid sequence using a CRISPR system comprising
providing a first foreign nucleic acid encoding a guide RNA sequence including a spacer sequence complementary to a protospacer sequence in the nucleic acid sequence,
providing a second foreign nucleic acid encoding a Cas9 protein,
wherein the guide RNA sequence and the Cas9 protein are expressed,
wherein the guide RNA sequence and the Cas9 protein co-localize to the nucleic acid sequence and the Cas9 protein binds or cleaves the nucleic acid sequence in a site specific manner, and
wherein the rate at which the guide RNA regulates the binding or cleavage of the nucleic acid sequence can be controlled.

62. The method of claim 61, wherein the rate at which the guide RNA regulates the binding or cleavage of the nucleic acid sequence can be controlled by adding additional nucleotide sequence between the transcription start site and the scaffold of the guide RNA.

63. The method of claim 61, wherein the method targets the nucleic acid sequence in a cell.

64. The method of claim 61, wherein the method targets the nucleic acid sequence in vitro.

65. The method of claim 61, wherein the nucleic acid sequence encodes a self-targeting guide RNA including a spacer sequence and a protospacer adjacent motif (PAM) adjacent to the spacer sequence, wherein the spacer sequence is complementary to a protospacer sequence in the nucleic acid.

66. The method of claim 65, wherein the rate at which the self-targeting guide RNA regulates the binding or cleavage of the nucleic acid sequence can be controlled by adding additional nucleotide sequence between the transcription start site and the scaffold of the guide RNA.

67. The method of claim 66, wherein increasing the length of the additional nucleotide sequence between the transcription start site and the scaffold of the guide RNA reduces the rate at which the guide RNA regulates the binding or cleavage of the first foreign nucleic acid sequence and/or the target nucleic acid sequence.
